(19) United States
(12) Patent Application Publication
(10) Pub. No.: US 2022/0386881 A1
(43) Pub. Date: Dec. 8, 2022

(54) BIOLOGICAL SIGNAL MEASUREMENT DEVICE, METHOD, AND NON-TRANSITORY STORAGE MEDIUM STORING PROGRAM

(71) Applicant: OMRON HEALTHCARE Co., Ltd., Kyoto (JP)

(72) Inventors: Naomi MATSUMURA, Kyoto (JP); Yasuhiro KAWABATA, Kyoto (JP); Kenji FUJII, Kyoto (JP); Reiji FUJITA, Kyoto (JP); Akito ITO, Kyoto (JP); Yuki SAKAGUCHI, Kyoto (JP)

(21) Appl. No.: 17/819,813

(22) Filed: Aug. 15, 2022

Related U.S. Application Data
(63) Continuation of application No. PCT/JP2021/003509, filed on Feb. 1, 2021.

(30) Foreign Application Priority Data
Feb. 20, 2020 (JP) 2020-027153

(51) Int. Cl. A61B 5/0245; A61B 5/024; A61B 5/00; A61B 5/318

(52) U.S. Cl. CPC A61B 5/0245; A61B 5/02416; A61B 5/7246; A61B 5/318; A61B 2560/0209; A61B 2562/06

(57) ABSTRACT

A device can be used for a long period of time without increasing the size and weight, and a biological signal is surely measured. An aspect of the present invention includes acquiring, from a first sensor, a first biological signal related to a heartbeat of a subject, acquiring, from a second sensor, a second biological signal related to the heartbeat of the subject, detecting a first feature from the first biological signal acquired, setting a light emission control pattern based on a detection timing of the first feature and information indicating time correlation between the first biological signal and the second biological signal, and driving a light emitting element of the second sensor to perform intermittent light emission based on the light emission control pattern set.

### CROSS-REFERENCE TO RELATED APPLICATIONS

This application is the U.S. national stage application filed pursuant to 35 U.S.C. 365(c) and 120 as a continuation of International Patent Application No. PCT/JP2021/003509, filed Feb. 1, 2021, which application claims priority to Japanese Patent Application No. 2020-027153, filed Feb. 20, 2020, which applications are incorporated herein by reference in their entireties.

### TECHNICAL FIELD

An aspect of the present invention relates to a biological signal measurement device, method, and a non-transitory storage medium storing a program for measuring a biological signal of a person, for example.

### BACKGROUND

For example, a pulse wave is known as one of biological signals. The pulse wave is a periodic waveform signal generated by the pulsation of the aorta in response to the heartbeat. Pulse wave velocity (PWV) flowing through the artery is correlated with volume elasticity of the blood vessel. The volume elasticity increases as blood pressure increases, and thus the blood pressure as well as the progress of arteriosclerosis can be estimated by obtaining the pulse wave velocity. The pulse wave velocity can be obtained by measuring pulse transit time (PTT) that is time for a pulse wave to transit between two different points on the artery, for example.

As described in Patent Document 1, a known technique of measuring the pulse transit time (PTT) calculates the pulse transit time in measuring the blood pressure based on outputs from an electrocardiogram (ECG) sensor attached to a person's body and a photoelectric sensor applying plethysmography (PPG) attached to the person's ear. As described in Patent Document 2, another known technique also calculates pulse transit time in measuring the blood pressure based on the pulse wave measured by PPG sensors disposed at two different points on the artery. Citations: Document 1, JP 5984088 B; and, Document 2 JP H7-327940.

### SUMMARY OF INVENTION

### Technical Problem

Unfortunately, the PPG sensor used for measuring the pulse transit time typically uses a light emitting diode (LED) as a light emitting element and thus consumes a larger amount of power than other biological sensors, such as ECG sensors. Thus, when the blood pressure is measured continuously during sleep (for eight hours for example) by using a blood pressure monitor using, for example, the PPG sensor, the measurement throughout the target measurement period may not be performed due to battery capacity shortage. To eliminate such a drawback, the battery capacity is increased, but this requires the battery to be larger and heavier resulting in the device being bulky and reduces the advantage of a wearable blood pressure monitor.

The present invention has been made in view of the above, and an aspect of the present invention is to provide a technique enabling the device to be used for a long period of time without increasing the size and weight and enabling a biological signal to be surely measured. Whereas there and other objects, features, and advantages of the present disclosure will become readily apparent upon a review of the following detailed description of the disclosure, in view of the drawings and appended claims.

### Solution to Problem

According to an aspect of a biological signal measurement device or a biological signal measurement method according to the present invention, a first biological signal that is related to a heartbeat of a subject is acquired from a first sensor, a second biological signal that is related to the heartbeat of the subject is acquired from a second sensor that uses a light emitting element, a first feature is detected from the first biological signal acquired, and the light emitting element of the second sensor is driven to perform intermittent light emission based on a detection timing of the first feature and information indicating time correlation between the first biological signal and the second biological signal.

### Advantageous Effects of Invention

According to an aspect of the present invention, the light emitting element of the second sensor is driven to perform the intermittent light emission. Thus, the power consumption can be reduced compared to the case of making the light emitting element continuously emit light. The light emitting period of the intermittent light emission driven is synchronized with the detection timing of a feature of the first biological signal measured from the same subject and is set based on the time correlation between the first biological signal and the second biological signal, whereby a feature of the second biological signal can be surely detected without fail.

An aspect of the present invention can provide a technique enabling the device to be used for a long period of time without increasing the size and weight and enabling a biological signal to be surely measured.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is a diagram illustrating an example of an overall configuration of a blood pressure measurement device that is a first embodiment of a biological signal measurement device according to the present invention;

FIG. 2 is a diagram illustrating an example of a configuration on a front surface side of an attachment unit of the blood pressure measurement device illustrated in FIG. 1;

FIG. 3 is a diagram illustrating an example of a configuration on a rear surface side of the attachment unit of the blood pressure measurement device illustrated in FIG. 1;

FIG. 4 is a cross-sectional view illustrating an example of a state in which the attachment unit of the blood pressure measurement device illustrated in FIG. 1 is attached to an upper arm part of a subject;

FIG. 5 is a block diagram illustrating an example of a hardware configuration of the blood pressure measurement device illustrated in FIG. 1;

FIG. 6 is a block diagram illustrating an example of a software configuration of the blood pressure measurement device illustrated in FIG. 1;

FIG. 7 is a flowchart illustrating a former half of procedure and content of processing executed by a blood pressure measurement unit of the blood pressure measurement device illustrated in FIG. 6;

FIG. 8 is a flowchart illustrating a latter half of procedure and content of processing executed by the blood pressure measurement unit of the blood pressure measurement device illustrated in FIG. 6;

FIG. 9 is a waveform diagram illustrating a first operation example of the blood pressure measurement device according to the first embodiment of the present invention;

FIG. 10 is a waveform diagram illustrating a second operation example of the blood pressure measurement device according to the first embodiment of the present invention;

FIG. 11 is a waveform diagram illustrating a third operation example of the blood pressure measurement device according to the first embodiment of the present invention;

FIG. 12 is a waveform diagram illustrating a fourth operation example of the blood pressure measurement device according to the first embodiment of the present invention;

FIG. 13 is a waveform diagram illustrating a fifth operation example of the blood pressure measurement device according to the first embodiment of the present invention;

FIG. 14 is a waveform diagram illustrating a sixth operation example of the blood pressure measurement device according to the first embodiment of the present invention;

FIG. 15 is a diagram illustrating an example of a configuration on a rear surface side of an attachment unit of a blood pressure measurement device that is a second embodiment of the biological signal measurement device according to the present invention;

FIG. 16 is a block diagram illustrating an example of a hardware configuration of the blood pressure measurement device according to the second embodiment of the present invention;

FIG. 17 is a block diagram illustrating an example of a software configuration of the blood pressure measurement device according to the second embodiment of the present invention;

FIG. 18 is a flowchart illustrating a former half of procedure and content of processing executed by a blood pressure measurement unit of the blood pressure measurement device illustrated in FIG. 17;

FIG. 19 is a flowchart illustrating a latter half of procedure and content of processing executed by the blood pressure measurement unit of the blood pressure measurement device illustrated in FIG. 17;

FIG. 20 is a waveform diagram illustrating a first operation example of the blood pressure measurement device according to the second embodiment of the present invention;

FIG. 21 is a waveform diagram illustrating a second operation example of the blood pressure measurement device according to the second embodiment of the present invention;

FIG. 22 is a diagram illustrating an example of a configuration on a rear surface side of an attachment unit of a blood pressure measurement device that is a third embodiment of the biological signal measurement device according to the present invention;

FIG. 23 is a block diagram illustrating a hardware configuration of the blood pressure measurement device according to the third embodiment of the present invention;

FIG. 24 is a block diagram illustrating a software configuration of the blood pressure measurement device according to the third embodiment of the present invention;

FIG. 25 is a flowchart illustrating a former half of procedure and content of processing executed by a blood pressure measurement unit of the blood pressure measurement device illustrated in FIG. 24;

FIG. 26 is a flowchart illustrating a latter half of procedure and content of processing executed by the blood pressure measurement unit of the blood pressure measurement device illustrated in FIG. 24; and,

FIG. 27 is a waveform diagram illustrating an operation example of the blood pressure measurement device according to the third embodiment of the present invention.

### DESCRIPTION OF EMBODIMENTS

Embodiments according to one aspect of the present invention will be described below based on the drawings. However, the embodiments described below are merely illustrative of the present invention in all respects. It should be appreciated that like drawing numbers on different drawing views identify identical, or functionally similar, structural elements of the invention. While the present invention is described with respect to what is presently considered to be the preferred aspects, it is to be understood that the invention as claimed is not limited to the disclosed aspect. The present invention is intended to include various modifications and equivalent arrangements within the spirit and scope of the appended claims. Furthermore, it is understood that this invention is not limited to the particular methodology, materials and modifications described and as such may, of course, vary.

It is also understood that the terminology used herein is for the purpose of describing particular aspects only and is not intended to limit the scope of the present invention, which is limited only by the appended claims. Unless defined otherwise, all technical and scientific terms used herein have the same meaning as commonly understood to one of ordinary skill in the art to which this invention belongs. Although any methods, devices or materials similar or equivalent to those described herein can be used in the practice or testing of the invention, the preferred methods, devices, and materials are now described.

### First Embodiment—Configuration Example

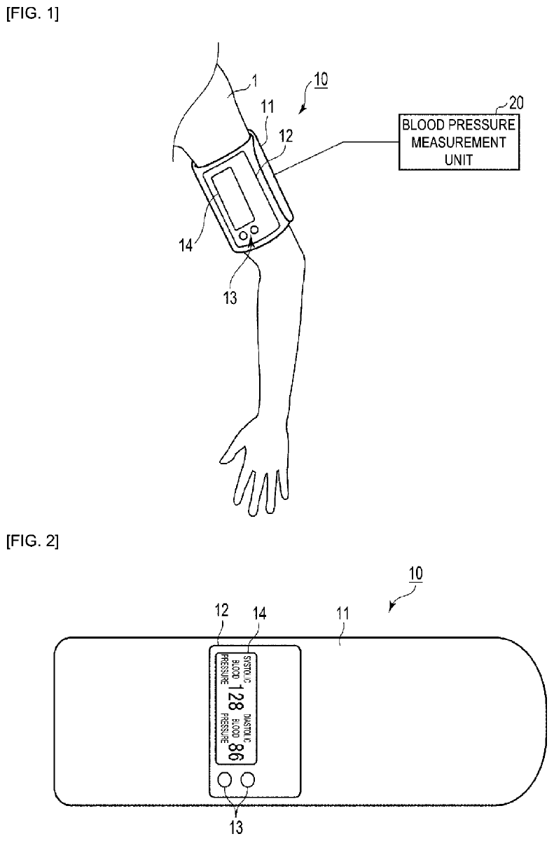

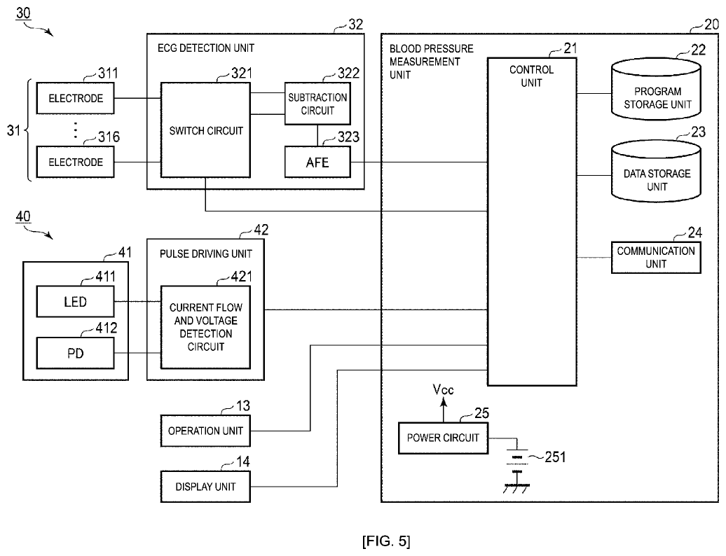

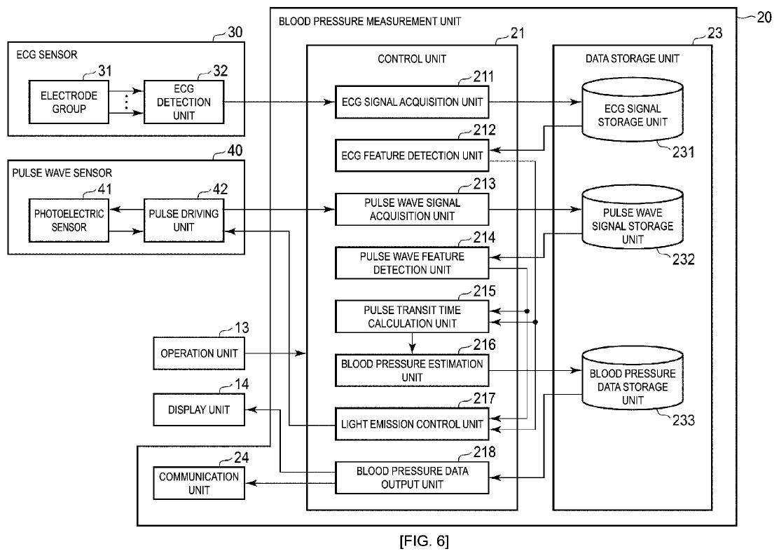

(1) Overall Configuration of Device. FIG. 1 is a diagram illustrating an overall configuration of a blood pressure measurement device that is a first embodiment of a biological signal measurement device according to the present invention. FIGS. 5 and 6 are block diagrams respectively illustrating the hardware configuration and the software configuration of the blood pressure measurement device illustrated in FIG. 1. The blood pressure measurement device according to the first embodiment includes an attachment unit 10 and a blood pressure measurement unit 20 to be connected to the attachment unit 10. FIG. 1 illustrates a case where the attachment unit 10 and the blood pressure measurement unit 20 is separately configured, but the blood pressure measurement unit 20 and the attachment unit 10 may be integrally provided to function the blood pressure measurement device as a so-called wearable device.

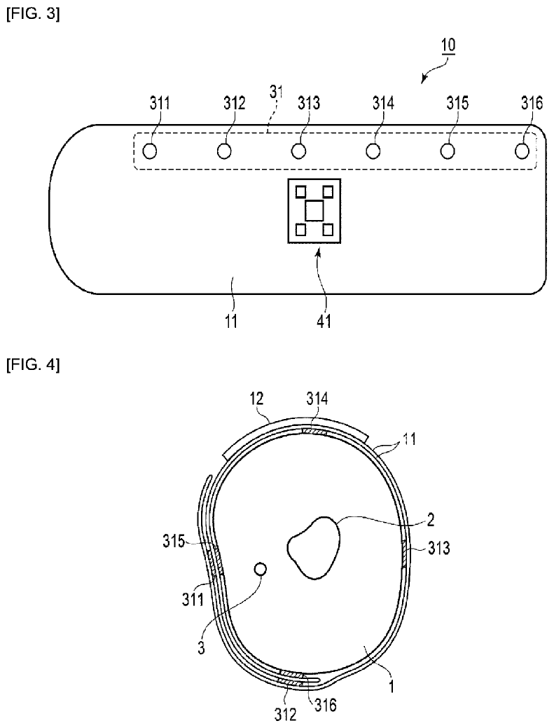

(2) Attachment Unit 10. The attachment unit 10 is attached to an upper arm part 1 of a subject as illustrated in FIG. 1. FIG. 2 illustrates a configuration example on a front surface side of the attachment unit 10, and FIG. 3 illustrates a configuration example on a rear surface side of the attachment unit 10.

The attachment unit 10 includes a belt portion 11 formed of, for example, flexible resin or fiber, and includes an attachment unit circuit unit 12 disposed on a front surface side of the belt portion 11. The attachment unit circuit unit 12 includes an operation unit 13, a display unit 14, an electrocardiographic (ECG) detection unit 32 of an ECG sensor 30 described below, and a pulse driving unit 42 of a pulse wave sensor 40.

The operation unit 13 includes, for example, a push button switch, and is used to input an instruction to start/end the blood pressure measurement, an instruction to display or transmit blood pressure data measured, and the like. The display unit 14 uses, for example, a liquid crystal or organic electro luminescence (EL) as a display device and is used for displaying blood pressure data measured and the like. Note that the operation unit 13 and the display unit 14 may be formed by a tablet device with a sheet for touch panel disposed on a display screen of the display unit.

On the other hand, as illustrated in FIG. 3, an electrode group 31 of the ECG sensor 30 is arranged on the rear surface side of the belt portion 11 in the longitudinal direction of the belt portion 11. The electrode group 31 includes a plurality of (six in this example) electrodes 311 to 316 arranged at an equal interval and is brought in contact with the skin of the subject to detect an ECG signal. As illustrated in FIG. 3, the disposed position of the electrode group 31 in a width direction of the belt portion 11 is set to be close to a shoulder of the subject. This is for enabling the ECG sensor 30 to detect the ECG signal at a position close to the heart of the subject as much as possible.

As illustrated in FIG. 5, the ECG detection unit 32 of the ECG sensor 30 includes a switch circuit 321, a subtraction circuit 322, and an analog front end (AFE) 323. The switch circuit 321 selects two of the six electrodes 311 to 316 to be connected to the subtraction circuit 322, based on a switching control signal output from a control unit 21 of the blood pressure measurement unit 20 described below. The subtraction circuit 322 includes, for example, an instrumentation amplifier, and outputs a potential difference between signals output from the two electrodes selected by the switch circuit 321 as described above. The AFE 323 includes, for example, a low-pass filter (LPF), an amplifier, and an analog-to-digital converter. The LPF removes unwanted noise component from the potential difference signal output from the subtraction circuit 322 as described above. The resultant signal is amplified by the amplifier, and then converted into a digital signal by the analog-to-digital converter. This digital signal obtained by the conversion is output as the ECG signal to the blood pressure measurement unit 20.

On the rear surface side of the belt portion 11, a photoelectric sensor 41 of the pulse wave sensor 40 is disposed at a substantially center portion in the longitudinal direction and the width direction of the belt portion 11. The photoelectric sensor 41 includes a light emitting diode (LED) 411 serving as a light emitting element, and a photo diode (PD) 412 serving as a light receiving element. The LED 411 emits light onto a skin surface of the upper arm part 1. The PD 412 receives reflected light that is the emitted light reflected on the skin surface. An electrical signal corresponding to the intensity of the light thus received is output to the pulse driving unit 42.

The pulse driving unit 42 of the pulse wave sensor 40 includes a current flow and voltage detection circuit 421. The current flow and voltage detection circuit 421 drives the LED 411 for the intermittent or continuous light emission, based on a light emission control signal output from the control unit 21 of the blood pressure measurement unit 20. Of these, the control operation for the intermittent light emission will be described below in detail. The current flow and voltage detection circuit 421 removes a noise component from the electrical signal output from the PD 412, amplifies the resultant signal to a predetermined level, converts the signal into a digital signal, and outputs the pulse wave signal including the digital signal thus converted to the blood pressure measurement unit 20.

Although not illustrated, a loop surface member and a hook surface member forming a surface fastener are attached respectively on the front surface side and the rear surface side of the belt portion 11. With the surface fastener, the attachment unit 10 is fixed with the belt portion 11 wound in a circumferential direction of the upper arm part 1 of the subject. FIG. 4 is a cross-sectional view illustrating an example of a state in which the attachment unit 10 is attached to the upper arm part 1.

(3) Blood Pressure Measurement Unit. The blood pressure measurement unit 20 includes the control unit 21 including a hardware processor such as a central processing unit (CPU), and has a program storage unit 22, a data storage unit 23, and a communication unit 24 connected to the control unit 21. The blood pressure measurement unit 20 includes a power circuit 25.

The communication unit 24 is used to transmit, for example, the measured blood pressure data to an information terminal, which is not illustrated, under the control by the control unit 21. A communication interface to be used includes an interface employing a small power data communication standard, such as Bluetooth (registered trademark). For example, a smartphone or a personal computer is used as the information terminal.

The power circuit 25 generates a required power supply voltage Vcc based on the output of a battery 251 and supplies the generated power supply voltage Vcc to each unit of the blood pressure measurement unit 20 and to the attachment unit circuit unit 12 of the attachment unit 10.

The program storage unit 22, for example, includes a storage medium that is a combination of a non-volatile memory writing and reading to and from which can be performed as needed, such as a hard disk drive (HDD) or a solid state drive (SSD), and a non-volatile memory such as a read only memory (ROM), and stores programs necessary for executing various kinds of control processing according to an embodiment of the present invention, as well as middleware such as an operating system (OS).

The data storage unit 23, for example, includes a storage medium that is a combination of a non-volatile memory writing and reading to and from which can be performed as needed, such as an HDD or an SSD and a volatile memory such as a random access memory (RAM), and includes an ECG signal storage unit 231, a pulse wave signal storage unit 232, and a blood pressure data storage unit 233, as main storage areas for implementing the first embodiment of the present invention.

The ECG signal storage unit 231 is used to chronologically store the ECG signals output from the ECG sensor 30. The pulse wave signal storage unit 232 is used to chronologically store the pulse wave signals output from the pulse wave sensor 40. The blood pressure data storage unit 233 is used to store blood pressure data for each heart rate estimated by the control unit 21 as described below.

The control unit 21 includes an ECG signal acquisition unit 211, an ECG feature detection unit 212, and a pulse wave signal acquisition unit 213, a pulse wave feature detection unit 214, and a pulse transit time calculation unit 215, a blood pressure estimation unit 216, a light emission control unit 217, and a blood pressure data output unit 218, as processing functions for implementing the first embodiment of the present invention. Any of these processing units 211 to 218 is implemented by the hardware processor of the control unit 21 executing a program stored in the program storage unit 22.

The ECG signal acquisition unit 211 executes processing of acquiring the ECG signals output from the ECG detection unit 32 of the ECG sensor 30, and temporarily and chronologically storing the acquired ECG signals in the ECG signal storage unit 231. The ECG feature detection unit 212 executes processing of reading the ECG signal from the ECG signal storage unit 231, and detecting from the ECG signal, an R-wave peak RP per heart rate that is one of the features of the ECG signal.

The pulse wave signal acquisition unit 213 executes processing of acquiring pulse wave signals output from the pulse driving unit 42 of the pulse wave sensor 40, and temporarily and chronologically storing the pulse wave signals in the pulse wave signal storage unit 232. The pulse wave feature detection unit 214 executes processing of reading the pulse wave signal from the pulse wave signal storage unit 232 and detecting rising (pulse wave rising) PS per heart rate that is one of the features of the pulse wave signal.

The pulse transit time calculation unit 215 executes processing of calculating pulse transit time (PTT) per heart rate based on a time difference between the R-wave peak RP detected by the ECG feature detection unit 212 and the pulse wave rising PS detected by the pulse wave feature detection unit 214. The blood pressure estimation unit 216 executes processing of estimating a blood pressure value corresponding to the pulse transit time (PTT) thus calculated, by using a conversion table indicating the relationship between PTT and blood pressure values stored in advance in the data storage unit 23, or by using a conversion formula, for example.

The light emission control unit 217 provides the pulse driving unit 42 with the light emission control signal for driving the LED 411 of the pulse wave sensor 40 to perform intermittent light emission and has the following processing functions, for example.

(1) Setting a preparation mode before a blood pressure measurement operation starts and estimating the time correlation between the ECG signal and the pulse wave signal in the preparation period set in advance. For example, the PTT is calculated for each of a plurality of heart rates within the preparation period, and an average value thereof is calculated. Then, based on the calculated PTT average value, a light emission control pattern defining a light emitting period and a turning-off period of the LED 411 is set. The light emitting period is set to at least include a predetermined period before and after the pulse wave rising PS of the pulse wave signal to include the pulse wave rising PS.

The length of the light emitting period may be set based on the longest PTT value obtained within the preparation period, instead of the PTT average value. With this configuration, the pulse wave rising PS of the pulse wave signal can be detected with a high possibility, even when a certain factor leads to a longer heart rate interval.

(2) Generating, each time the R-wave peak RP of the ECG signal is detected under a blood pressure measurement mode after the end of the preparation mode, the light emission control signal for driving the LED 411 of the pulse wave sensor 40 to perform intermittent light emission under the light emission control pattern set under the preparation mode in synchronization with the detection timing of the R-wave peak RP. Then, the pulse driving unit 42 of the pulse wave sensor 40 is provided with the generated light emission control signal.

When a display request for the blood pressure data is input through the operation unit 13, the blood pressure data output unit 218 executes processing of reading the blood pressure data from the blood pressure data storage unit 233 and displaying the blood pressure data on the display unit 14. When a transmission request for the blood pressure data is input through the operation unit 13, the blood pressure data output unit 218 executes processing of reading the blood pressure data from the blood pressure data storage unit 233 and transmitting the blood pressure data from the communication unit 24 to the information terminal set in advance as a transmission destination.

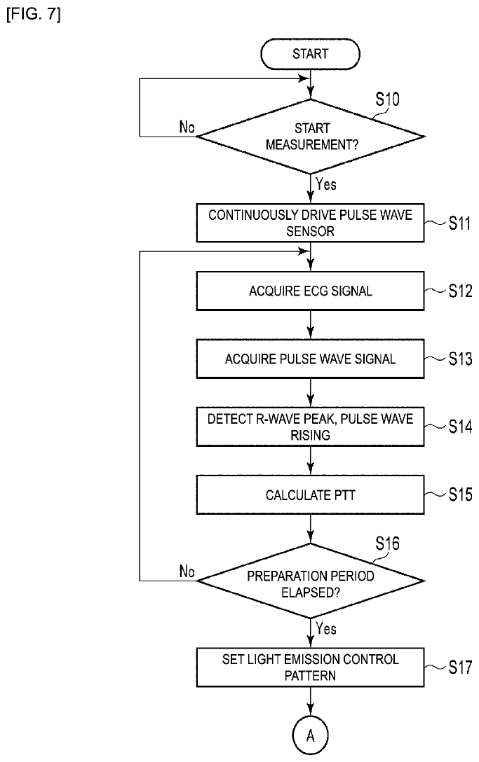

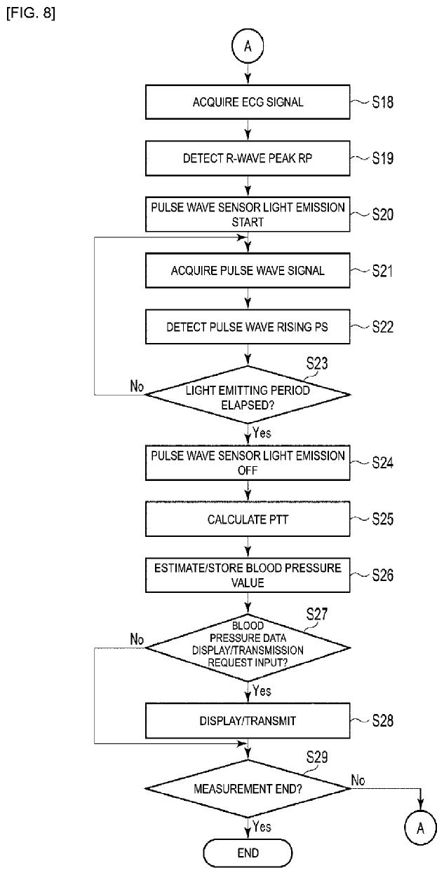

Operation Example. Next, operations of the blood pressure measurement device configured as described above will be described. Note that this example describes a case where a subject measures a change in the subject's blood pressure while sleeping, for example. FIGS. 7 and 8 are flowcharts illustrating a procedure and contents of processing executed by the control unit 21 of the blood pressure measurement unit 20.

(1) Preparation Mode. First of all, the subject winds the belt portion 11 of the attachment unit 10 around the subject's upper arm part 1 and fixes the attachment unit 10 using the surface fastener, with the belt rear surface side being in contact with the skin surface of the upper arm part 1. In this state, the operation unit 13 provided to the attachment unit 10 is operated to input a measurement start request. This measurement start request also serves as a power ON signal.

In step S10, the blood pressure measurement unit 20 monitors the input of the measurement start request. In this state, when the measurement start request is input from the attachment unit 10, the power circuit 25 operates under the control by the control unit 21 and starts supplying the power supply voltage Vcc to each unit of the device. This results in the blood pressure measurement unit 20 and the attachment unit 10 being in an operating state.

Upon being in the operating state, the blood pressure measurement unit 20 first generates a continuous light emission control signal and provides the pulse driving unit 42 of the pulse wave sensor 40 with the continuous light emission control signal generated under the control by the light emission control unit 217 in step S11. As a result, the LED 411 is driven by the pulse driving unit 42 to continuously emit light. Thus, the pulse wave signals detected by the pulse wave sensor 40 are continuously output.

In this state, in step S12, the blood pressure measurement unit 20 makes the ECG signal acquisition unit 211 acquire the ECG signals output from the ECG sensor 30 and makes the ECG signal storage unit 231 chronologically store the ECG signals. In step S13, the blood pressure measurement unit 20 makes the pulse wave signal acquisition unit 213 acquire the pulse wave signals output from the pulse wave sensor 40 and makes the pulse wave signal storage unit 232 chronologically store the pulse wave signals.

Then, in step S14, the blood pressure measurement unit 20 makes the ECG feature detection unit 212 read the ECG signal from the ECG signal storage unit 231, and detect the R-wave peak RP that is one of the features of the ECG signal, and further makes the pulse wave feature detection unit 214 read the pulse wave signal from the pulse wave signal storage unit 232 and detect the pulse wave rising PS that is one of the features of the pulse wave signal.

Then, in step S15, the blood pressure measurement unit 20 makes the pulse transit time calculation unit 215 calculate the time difference between the detection timings of the R-wave peak RP and the pulse wave rising PS detected in a single heart rate cycle and store the PTT in a PTT data storage unit (not illustrated) in the data storage unit 23 as the pulse transit time (PTT) in the single heart rate cycle.

Then, in step S16, the blood pressure measurement unit 20 monitors whether the preparation period set in advance elapses. The processing returns to step S11 when the preparation period has not elapsed yet. Then, the processing of calculating the PTT for each heart rate in steps S11 to S15 is repeatedly executed. The preparation period is set to a time period corresponding to 10 to 20 heart rates which is an example of an average time period required for stabilizing the heart rate. However, the length of the preparation period is not limited to the time period.

On the other hand, when the preparation period elapses, the blood pressure measurement unit 20 makes the LED 411 of the pulse wave sensor 40 in the continuous light emitting state to temporarily return to the turning-off state. Then, in step S17, for example, the light emission control unit 217 calculates the average value of PTTs of the respective heart rates calculated in the preparation period, and sets the light emission control pattern, that is, the lengths of the light emitting period and the turning-off period, for driving the LED 411 of the pulse wave sensor 40 to perform the intermittent light emission, based on the PTT average value calculated. A typical operation example based on the light emission control pattern will be described below in detail.

(2) Blood Pressure Measurement Mode. When the setting of the light emission control pattern under the preparation mode ends, the blood pressure measurement unit 20 starts an operation of controlling the blood pressure measurement for each heart rate, as described below.

Specifically, in step S18, the blood pressure measurement unit 20 first makes the ECG signal acquisition unit 211 acquire the ECG signals output from the ECG sensor 30 and makes the ECG signal storage unit 231 chronologically store the ECG signals. Then, in step S19, the ECG feature detection unit 212 reads the ECG signal from the ECG signal storage unit 231, detects the R-wave peak RP from the ECG signal thus read, and stores the detection timing thereof in an ECG feature storage unit (not illustrated) in the data storage unit 23.

Next, in step S20, the blood pressure measurement unit 20 generates, in accordance with the light emission control pattern set in the preparation mode under the control by the light emission control unit 217, a light emission control signal for making the LED 411 of the pulse wave sensor 40 start emit light in synchronization with the detection timing of the R-wave peak RP and provides the pulse driving unit 42 of the pulse wave sensor 40 with the light emission control signal. As a result, the LED 411 of the pulse wave sensor 40 starts emitting light, and the pulse wave sensor 40 outputs the pulse wave signal of the subject.

In step S21, the blood pressure measurement unit 20 makes the pulse wave signal acquisition unit 213 acquire the pulse wave signals output from the pulse wave sensor 40 and makes the pulse wave signal storage unit 232 chronologically store the pulse wave signals. Then, in step S22, the pulse wave feature detection unit 214 reads the pulse wave signal from the pulse wave signal storage unit 232 and detects the pulse wave rising PS from the pulse wave signal. When the pulse wave rising PS is detected, the detection timing thereof is stored in a pulse wave feature storage unit (not illustrated) in the data storage unit 23.

Under the control by the light emission control unit 217, in step S23, the blood pressure measurement unit 20 monitors the end timing of the light emitting period defined by the light emission control pattern described above. When the light emitting period ends, in step S24, the light emission by the LED 411 of the pulse wave sensor 40 ends.

When the pulse wave rising PS is detected, in step S25, the blood pressure measurement unit 20 makes the pulse transit time calculation unit 215 calculate, as the PTT of the current heart rate, a time difference between the detection timing of the R-wave peak RP of the ECG signal that has been detected in step S19 and the detection timing of the pulse wave rising PS of the pulse wave signal detected in step S22 as described above. Then, in step S26, the blood pressure estimation unit 216 estimates the blood pressure value based on the PTT calculated and stores the estimated blood pressure value in the blood pressure data storage unit 233 in association with the detection timing of the R-wave peak RP, that is, identification information on the heart rate. As a result, the blood pressure value of a single heart rate of the subject is stored in the blood pressure data storage unit 233.

The blood pressure measurement unit 20 makes the blood pressure data output unit 218 monitor the input of the blood pressure data display/transmission request in step S27, while executing the processing for the blood pressure measurement described above. For example, when the subject operates the operation unit 13 for the display/transmission request, under the control by the blood pressure data output unit 218, the blood pressure data is read from the blood pressure data storage unit 233 to be displayed on the display unit 14 or transmitted to the information terminal from the communication unit 24 in step S28.

The blood pressure measurement unit 20 monitors the input of a measurement end request in step S29, while executing the processing for the blood pressure measurement. In this state, when the subject operates the operation unit 13 for the measurement end request, for example, the blood pressure measurement unit 20 ends the processing for the blood pressure measurement and stops the supply of the power supply voltage Vcc to each unit from the power circuit 25.

The blood pressure data stored in the blood pressure data storage unit 233 is retained even after the end of the power supply. For example, the light emission control pattern set by the light emission control unit 217 may be stored in the data storage unit 23 in association with the identification information on the subject. With this configuration, the blood pressure measurement to be performed again for the same subject can be immediately started based on the light emission control pattern corresponding to the subject.

Example of Typical Operation. Next, a typical operation example according to the first embodiment will be described. Note that the operation example is not limited to the following example, and various other operation examples are conceivable.

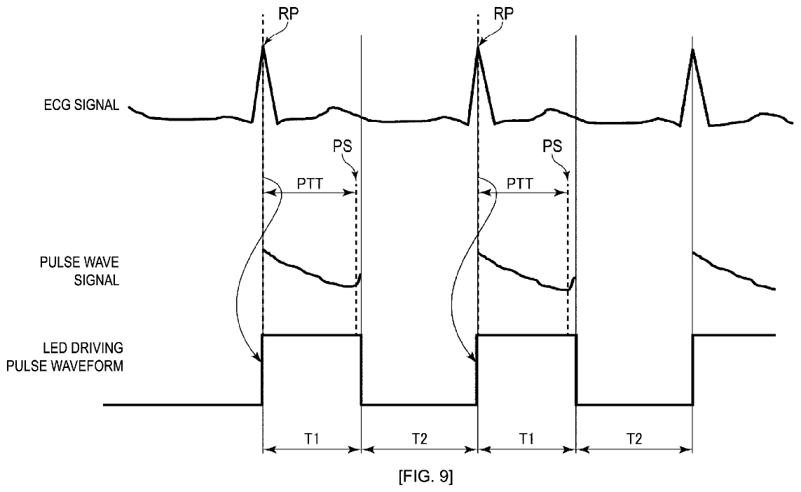

(1) First Operation Example. FIG. 9 is a signal waveform diagram illustrating a first operation example. Based on the PTT average value calculated as the time difference between the R-wave peak RP of the ECG signal and the pulse wave rising PS of the pulse wave signal for each heart rate in the preparation period, the blood pressure measurement unit 20 first sets the light emitting period to be a period T1 that is longer than the PTT average value by a predetermined time period, and sets the turning-off period to be a period T2 until the detection of the R-wave peak RP of the ECG signal of the next heart rate.

When the R-wave peak RP of the ECG signal is detected under the blood pressure measurement mode, the blood pressure measurement unit 20 makes the LED 411 of the pulse wave sensor 40 emit light starting from the detection timing of the R-wave peak RP. Then, the pulse wave sensor 40 operates to output the pulse wave signal. The blood pressure measurement unit 20 detects the pulse wave rising PS from the pulse wave signal output. Then, the time difference between the detection timing of the R-wave peak RP of the ECG signal and the detection timing of the pulse wave rising PS of the pulse wave signal is calculated as the PTT of a single heart rate, and the blood pressure value is estimated based on this PTT.

When the length of the light emitting period reaches the setting value T1 set for the light emitting period in the preparation mode during the light emission operation of the LED 411 of the pulse wave sensor 40, the blood pressure measurement unit 20 makes the LED 411 turn off. Then, this turning-off state is maintained until the R-wave peak RP of the ECG signal of the next heart rate is detected. Thereafter, each time the R-wave peak RP of the ECG signal is detected, the blood pressure measurement unit 20 operates the LED 411 of the pulse wave sensor 40 to perform the intermittent light emission operation in synchronization with the detection timing of the R-wave peak RP and repeats the processing of measuring the blood pressure value for each heart rate.

According to the first operation example, the LED 411 of the pulse wave sensor 40 performs the light emission operation only in the light emitting period T1 set in the preparation period in synchronization with the R-wave peak RP of the ECG signal for each heart rate. Thus, compared to the case of causing the LED 411 of the pulse wave sensor 40 to constantly emit light, the power consumed by the LED 411 of the pulse wave sensor 40 can be reduced, allowing the blood pressure to be continuously measured throughout the sleeping period without the battery 251 having a large capacity.

Furthermore, according to the first operation example, the light emitting period T1 of the LED 411 is set to a value that is longer than the PTT value by the predetermined length starting from the detection timing of the R-wave peak RP, allowing the pulse wave rising PS of the pulse wave signal to be detected without fail. Thus, the blood pressure for each heart rate can be measured without a lack of data.

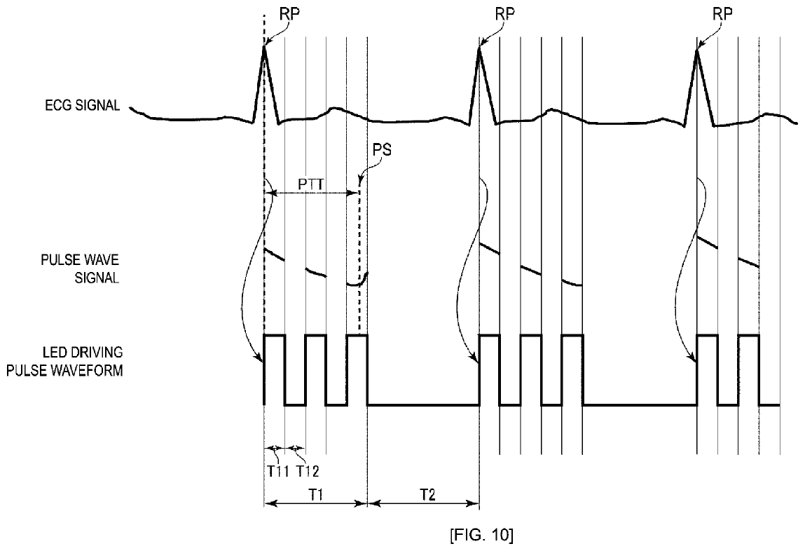

(2) Second Operation Example. FIG. 10 is a signal waveform diagram illustrating a second operation example.

In this example, the blood pressure measurement unit 20 sets the length of the light emitting period to T1 as in the first operation example illustrated in FIG. 9 and sets an intermittent turning-off period in the light emitting period T1. A ratio between light emitting period and turning-off period in the light emitting period T1, that is, the duty ratio is set to be 50%, for example, but may be of any value exceeding 0% but not exceeding 100%.

According to the second operation example, the LED 411 further operates to perform an intermittent light emission operation during the period T1 set for detecting the pulse wave rising PS of the pulse wave signal, further reducing the total light emitting period of the LED 411. As a result, the power consumption of the battery 251 is further reduced, whereby the blood pressure can be continuously measured for a longer period of time.

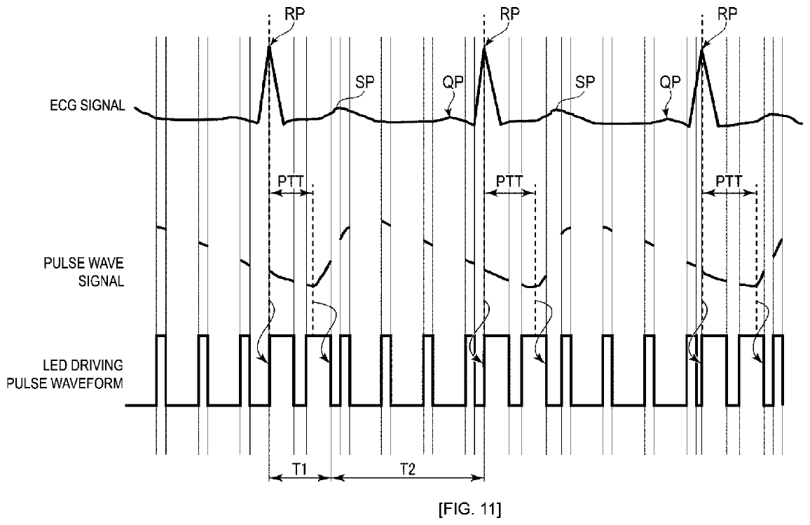

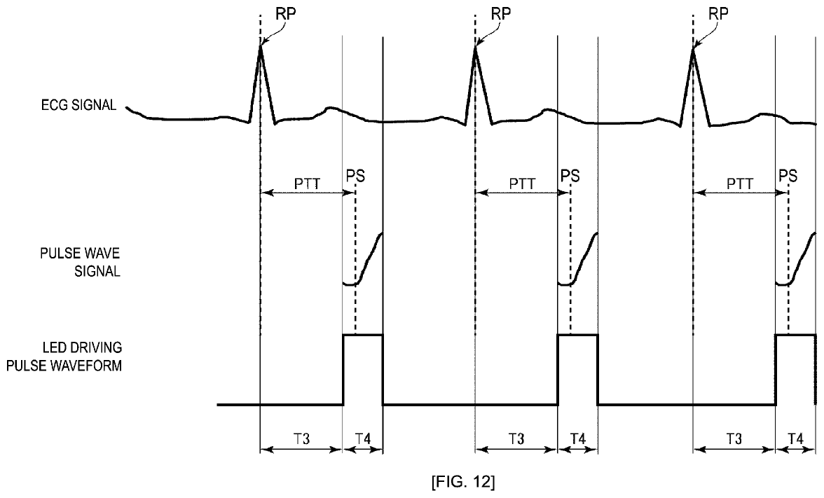

(3) Third Operation Example. FIG. 11 is a signal waveform diagram illustrating a third operation example. In this example, the blood pressure measurement unit 20 sets the length of the light emitting period to T1 as in the first operation example illustrated in FIG. 9 and sets a turning-off period in the light emitting period T1. Furthermore, an intermittent light emitting period is set in the turning-off period T2 other than the light emitting period T1. A ratio between light emitting period and the turning-off period in the turning-off period T2, that is, the duty ratio is set to be 25%, for example, but may be of any value exceeding 0% but not exceeding 100%.

According to the third operation example, with the turning-off period set in the light emitting period T1, the power consumption of the battery 251 can be further reduced from that in the first operation example. Furthermore, setting the intermittent light emitting period in the turning-off period T2 allows the pulse wave signal to be detected intermittently even in the turning-off period T2 as illustrated in FIG. 11. Thus, even when the timing of the pulse wave rising PS is shifted due to a temporary change in the heart rate cycle, for example, such a shift can be detected with a higher possibility.

(4) Fourth Operation Example. FIG. 12 is a signal waveform diagram illustrating a fourth operation example. In this example, the blood pressure measurement unit 20 first sets a standby period T3 starting from the detection timing of the R-wave peak RP of the ECG signal, and then sets a light emitting period T4 after the elapse of the standby period T3.

The standby period T3 and the light emitting period T4 are set as follows, for example. Specifically, the blood pressure measurement unit 20 obtains the PTT average or minimum value under the preparation mode, and also obtains the average value or the maximum value of the shift in the detection timing of the pulse wave rising PS. Then, the standby period T3 and the light emitting period T4 are set based on the above-described values thus obtained. For example, the standby period T3 is set so as not to include the pulse wave rising PS even if the timing of such rising is shifted to be earlier. The light emitting period T4 is set to include the pulse wave rising PS even if the timing of the rising changes.

According to the fourth operation example, for each heart rate, the standby period T3 starting from the detection timing of the R-wave peak RP of the ECG signal is set, and the light emitting period T4 after the elapse of the standby period T3 is set. Thus, the LED 411 of the pulse wave sensor 40 can perform the light emission operation limitedly in a period in which the pulse wave rising PS of the pulse wave signal is expected to be detected, allowing the light emitting period of the LED 411 for each heart rate to be further reduced. As a result, the power consumption of the battery can be further reduced, and the time period during which the blood pressure can be continuously measured can be further increased.

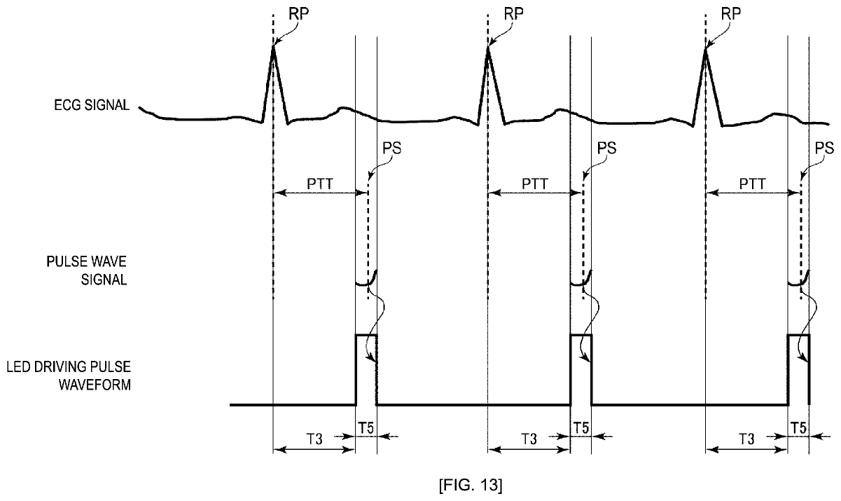

(5) Fifth Operation Example. FIG. 13 is a signal waveform diagram illustrating a fifth operation example. The fifth operation example is a further improved version of the fourth operation example, in which the end timing of the light emitting period for each heart rate is synchronized with the detection timing of the pulse wave rising PS.

Specifically, the blood pressure measurement unit 20 first sets, for each heart rate, the standby period T3 starting from the detection timing of the R-wave peak RP of the ECG signal. Then, when the standby period T3 elapses, the light emission starts. The light emission ends at a point when the pulse wave rising PS of the pulse wave signal of the next heart rate is detected thereafter.

According to the fifth operation example, a light emitting period T5 of the LED 411 for each heart rate ends at a point when the pulse wave rising PS of the pulse wave signal is detected. Thus, compared to the fourth operation example, the light emission operation period of the LED 411 of the pulse wave sensor can be further reduced, allowing the power consumption of the battery 251 to be further reduced and the continuous measurement time of the blood pressure to be extended.

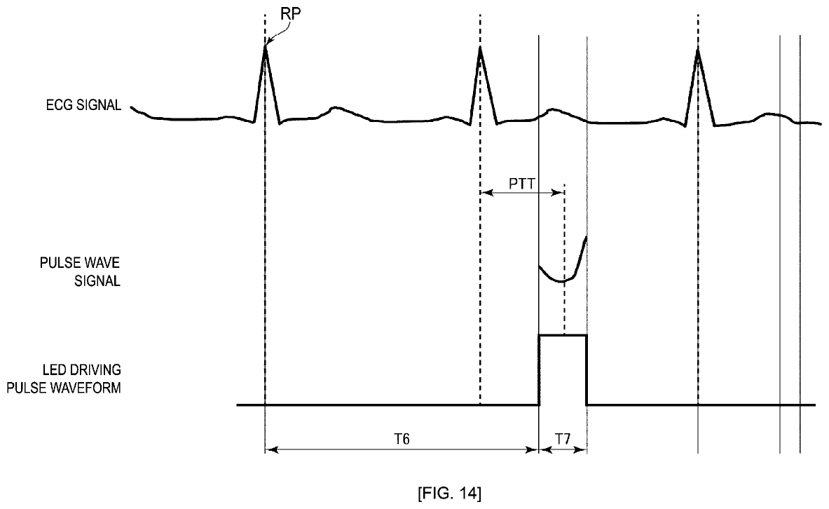

(6) Sixth Operation Example. FIG. 14 is a signal waveform diagram illustrating a sixth operation example. The sixth operation example is a further improved version of the fourth operation example. Specifically, when setting a standby period in synchronization with the detection timing of the R-wave peak RP of the ECG signal, the blood pressure measurement unit 20 sets a standby period T6 extended by a single heart rate cycle and sets a light emitting period T7 after the elapse of this standby period T6.

According to the sixth operation example, the light emission control in synchronization with the detection timing of the R-wave peak RP of the ECG signal can be implemented, with the start timing of the light emitting period T7 delayed by a single heart rate cycle. As a result, even when the control unit 21 has a low processing speed or a high processing load which is likely to lead to processing delay, the light emission control can be accurately performed without delay. Note that in this sixth operation example, the light emitting period T7 may end at a point when the pulse wave rising PS of the pulse wave signal of the next heart rate is detected.

Effect. In the first embodiment of the present invention as described in detail above, the LED 411 of the pulse wave sensor 40 performs the intermittent light emission operation, and the light emission control pattern for this intermittent light emission operation is synchronized with the detection timing of the R-wave peak RP of the ECG signal for each heart rate and is set based on the PTT indicating the time correlation between the ECG signal and the pulse wave signal.

Accordingly, it is possible to reduce the power consumed by the LED 411 of the pulse wave sensor 40 and reduce the power consumption of the battery 251, allowing the blood pressure for each heart rate of the subject to be continuously measured, for example, throughout the sleeping period without a large-capacity battery. The light emitting period of the LED 411 of the pulse wave sensor 40 is set based on the PTT calculated from the ECG signal obtained by the ECG sensor 30 and the pulse wave signal obtained from the pulse wave sensor 40, enabling the pulse wave rising PS of the pulse wave signal to be surely detected without fail. This allows the blood pressure for each heart rate to be measured without a lack of data.

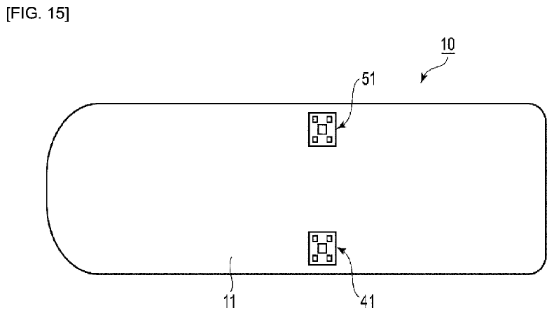

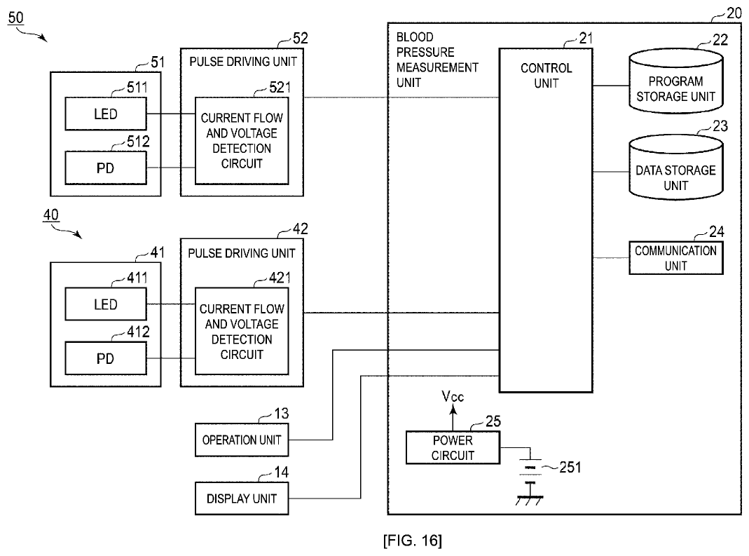

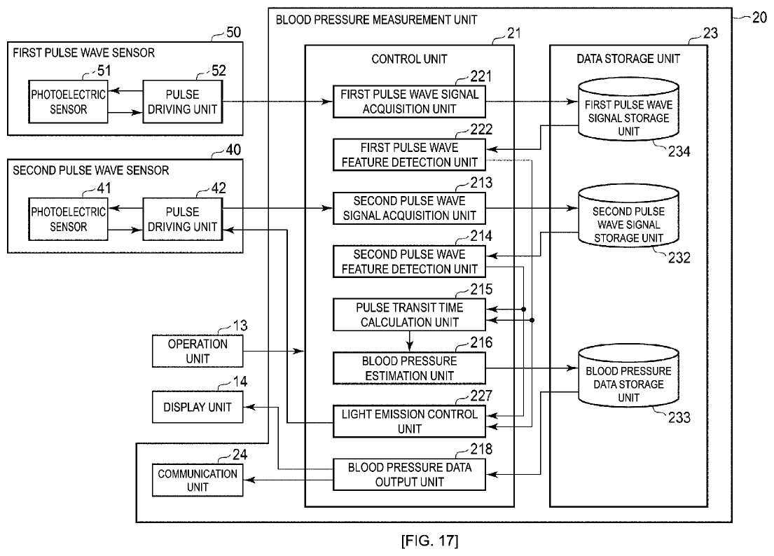

Second Embodiment; Configuration Example. FIG. 15 is a diagram illustrating a configuration on the rear surface side of the attachment unit 10 used in a blood pressure measurement device according to a second embodiment of the present invention. FIGS. 16 and 17 are block diagrams respectively illustrating the hardware configuration and the software configuration of the blood pressure measurement device illustrated. The same parts in FIGS. 15, 16, and 17 as those in FIGS. 3, 5, and 6 are denoted with the same reference numerals, and the detailed description thereof will be omitted.

(1) Attachment Unit. On the rear surface side of the belt portion 11 of the attachment unit 10, a photoelectric sensor 51 of a first pulse wave sensor 50 and the photoelectric sensor 41 of a second pulse wave sensor 40 are arranged separated from each other by a predetermined distance in the width direction of the belt portion 11. The positional relationship between the photoelectric sensors 41 and 51 is set to make the photoelectric sensor 51 of the first pulse wave sensor 50 arranged on the side closer to the heart of the subject and make the photoelectric sensor 41 of the second pulse wave sensor 40 arranged on the farther side. The photoelectric sensors 41 and 51 respectively include LEDs 411 and 511 serving as the light emitting elements, and PDs 412 and 512 serving as the light receiving elements.

The second pulse wave sensor 40 corresponds to the pulse wave sensor 40 described in the first embodiment, and photoelectric sensor 41 includes the LED 411 serving as the light emitting element and the PD 412 serving as the light receiving element. The pulse driving unit 42 drives the LED 411 to perform intermittent light emission, based on the light emission control signal output from the control unit 21 of the blood pressure measurement unit 20.

On the other hand, the first pulse wave sensor 50 is used in place of the ECG sensor 30 described in the first embodiment, and the photoelectric sensor 51 includes the LED 511 serving as the light emitting element and the PD 512 serving as the light receiving element. A pulse driving unit 52 drives the LED 511 to perform continuous light emission, using a current flow and voltage detection circuit 521. When the light emission control signal instructing the intermittent light emission is transmitted from the control unit 21, the pulse driving unit 52 drives the LED 511 to perform the intermittent light emission based on the light emission control signal.

As with the current flow and voltage detection circuit 421 of the second pulse wave sensor 40, the current flow and voltage detection circuit 521 of the first pulse wave sensor 50 removes a noise component from the electrical signal output from the PD 512, amplifies the resultant signal to a predetermined level, converts the signal into a digital signal, and outputs the pulse wave signal including the digital signal thus converted to the blood pressure measurement unit 20.

(2) Blood Pressure Measurement Unit. The data storage unit 23 includes a first pulse wave signal storage unit 234, a second pulse wave signal storage unit 232, and the blood pressure data storage unit 233 to implement the second embodiment of the present invention. The first pulse wave signal storage unit 234 is used to store a first pulse wave signal output from the first pulse wave sensor 50. The second pulse wave signal storage unit 232 corresponds to the pulse wave signal storage unit 232 described in the first embodiment and is used to store a second pulse wave signal output from the second pulse wave sensor 40. The blood pressure data storage unit 233 is used to store blood pressure data for each heart rate estimated by the control unit 21.

The control unit 21 includes a first pulse wave signal acquisition unit 221 and a first pulse wave feature detection unit 222 as processing functions replacing the ECG signal acquisition unit 211 and the ECG feature detection unit 212 described in the first embodiment. These processing units 221 and 222 are also implemented by the hardware processor of the control unit 21 executing a program stored in the program storage unit 22, as in the case of the other processing units 213 to 218.

The first pulse wave signal acquisition unit 221 executes processing of acquiring first pulse wave signals output from the pulse driving unit 52 of the first pulse wave sensor 50, and chronologically storing the first pulse wave signals in the first pulse wave signal storage unit 234. The first pulse wave feature detection unit 222 executes processing of reading the first pulse wave signal from the first pulse wave signal storage unit 234, and detecting from the first pulse wave signal, pulse wave rising PS1 per heart rate that is one of the features of the first pulse wave signal.

The pulse wave signal acquisition unit (herein referred to as a second pulse wave signal acquisition unit to be distinguished from the first pulse wave signal acquisition unit) 213 executes processing of acquiring pulse wave signals (referred to as a second pulse wave signal for the same reason) output from the pulse driving unit 42 of the pulse wave sensor (referred to as a second pulse wave sensor for the same reason) 40, and chronologically storing the second pulse wave signals in the pulse wave signal storage unit (referred to as a second pulse wave signal storage unit for the same reason) 232. The pulse wave feature detection unit (referred to as a second pulse wave feature detection unit for the same reason) 214 executes processing of reading the second pulse wave signal from the second pulse wave signal storage unit 232, and detecting from the second pulse wave signal, pulse wave rising PS2 that is one of the features of the second pulse wave signal.

The pulse transit time calculation unit 215 executes processing of calculating the pulse transit time (PTT) for each heart rate, based on the time difference between the first pulse wave rising PS1 detected by the first pulse wave feature detection unit 222 and the second pulse wave rising PS2 detected by the second pulse wave feature detection unit 214.

As in the first embodiment, the blood pressure estimation unit 216 executes processing of obtaining the blood pressure value corresponding to the pulse transit time (PTT) calculated, by using the conversion table indicating the relationship between the PTT and blood pressure values, or by using the conversion formula.

A light emission control unit 227 provides the pulse driving unit 42 with the light emission control signal for making the LED 411 of the second pulse wave sensor 40 perform intermittent light emission and has the following processing functions, for example.

(1) Setting a preparation mode before a blood pressure measurement operation starts and estimating the time correlation between the first pulse wave signal and the second pulse wave signal in the preparation period set in advance. For example, the PTT is calculated for each of a plurality of heart rates detected within the preparation period, and an average value thereof is calculated. Then, based on the calculated PTT average value, a light emission control pattern defining a light emitting period and a turning-off period of the LED 411 is set. The light emitting period is set to at least include a predetermined period before and after the pulse wave rising PS of the second pulse wave signal to include the pulse wave rising PS2.

The length of the light emitting period may be set based on the longest PTT value obtained within the preparation period, instead of the PTT average value. With this configuration, the pulse wave rising PS of the pulse wave signal can be detected with a high possibility, even when a certain factor leads to a longer heart rate interval.

(2) Generating, each time the pulse wave rising PS1 of the first pulse wave signal is detected under a blood pressure measurement mode after the end of the preparation mode, the light emission control signal for driving the LED 411 to perform intermittent light emission under the light emission control pattern set in the preparation mode in synchronization with the detection timing of the pulse wave rising PS1. Then, the pulse driving unit 42 of the second pulse wave sensor 40 is provided with the generated light emission control signal.

(3) When the LED 511 of the first pulse wave sensor 50 is driven to perform the intermittent light emission, a light emission control pattern opposed to the light emission control pattern for the LED 411 of the second pulse wave sensor 40 is set, and the pulse driving unit 52 of the first pulse wave sensor 50 is provided with the light emission control signal corresponding to the light emission control pattern set.

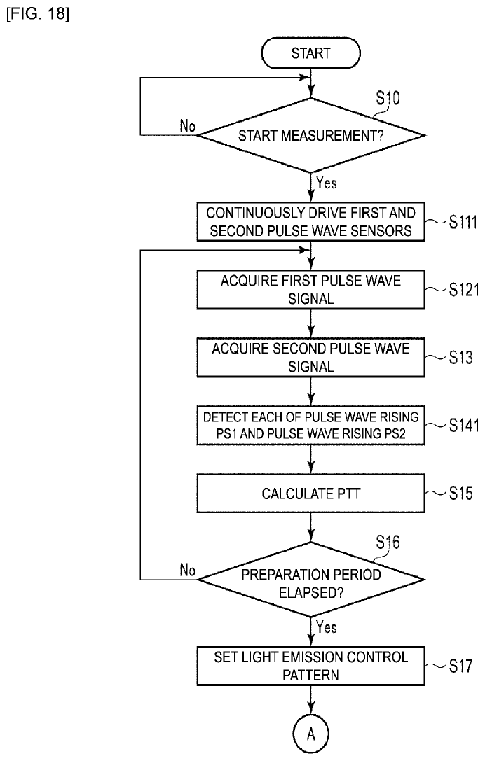

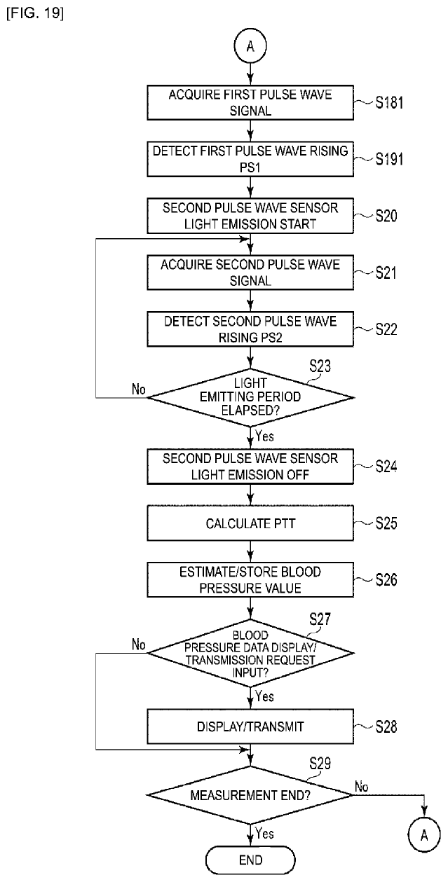

Operation Example. Next, operations of the blood pressure measurement device configured as described above will be described. FIGS. 18 and 19 are flowcharts illustrating a procedure and contents of processing executed by the control unit 21 of the blood pressure measurement unit 20. Note that in FIGS. 18 and 19, steps with the same contents of processing as those of the processing in FIGS. 7 and 8 described above will be described with the same reference numerals added.

(1) Preparation Mode. When the subject who has attached the attachment unit 10 to the subject's upper arm part 1 operates the operation unit 13 to input the measurement start request, the blood pressure measurement unit 20 detects the measurement start request in step S10. As a result, the power supply voltage Vcc is supplied to each unit of the device from power circuit 25. Thus, the blood pressure measurement unit 20 and the attachment unit 10 are in the operating state.

Upon being in the operating state, the blood pressure measurement unit 20 first generates a continuous light emission control signal and provides the continuous light emission control signal thus generated to the respective pulse driving units 52 and 42 of the first pulse wave sensor 50 and the second pulse wave sensor 40 under the control by the light emission control unit 227 in step S111. As a result, the LEDs 511 and 411 are driven by the pulse driving units 52 and 42 to perform continuous light emission. Thus, the first pulse wave sensor 50 and the second pulse wave sensor 40 continuously output the first pulse wave signal and the second pulse wave signal respectively.

In this state, in step S121, the blood pressure measurement unit 20 makes the first pulse wave signal acquisition unit 221 acquire the first pulse wave signals output from the first pulse wave sensor 50 and makes the first pulse wave signal storage unit 234 temporarily store the first pulse wave signals. In step S13, the blood pressure measurement unit 20 makes the second pulse wave signal acquisition unit 213 acquire the second pulse wave signals output from the second pulse wave sensor 40 and makes the second pulse wave signal storage unit 232 temporarily store the second pulse wave signals.

Then, in step S141, the blood pressure measurement unit 20 makes the first pulse wave feature detection unit 222 read the first pulse wave signal from the first pulse wave signal storage unit 234 and detect the pulse wave rising PS1 of the first pulse wave signal. Meanwhile, the second pulse wave feature detection unit 214 reads the second pulse wave signal from the second pulse wave signal storage unit 232 and detects the pulse wave rising PS2 of the second pulse wave signal.

Subsequently, in step S15, the blood pressure measurement unit 20 makes the pulse transit time calculation unit 215 calculate the time difference between the detection timing of the detected pulse wave rising PS1 of the first pulse wave signal and the detection timing of the pulse wave rising PS2 of the second pulse wave signal, and temporarily stores the calculated time difference in the data storage unit 23 as the pulse transit time (PTT) for the current heart rate.

Then, in step S16, the blood pressure measurement unit 20 monitors whether the preparation period set in advance elapses. The processing returns to step S111 when the preparation period has not elapsed yet. Then, the processing of calculating the PTT for each heart rate is repeatedly executed. The preparation period is set to a time period corresponding to 10 to 20 heart rates which is an example of an average time period required for stabilizing the heart rate. However, the length of the preparation period is not limited to the time period.

On the other hand, when the preparation period elapses, the blood pressure measurement unit 20 makes the LED 411 of the second pulse wave sensor 40 in the continuous light emitting state to temporarily return to the turning-off state. Then, in step S17, for example, the light emission control unit 227 calculates the average value of PTTs of the respective heart rates calculated in the preparation period, and sets the light emission control pattern, that is, the lengths of the light emitting period and the turning-off period, for driving the LED 411 of the second pulse wave sensor 40 to perform intermittent light emission, based on the PTT average value calculated.

(2) Blood Pressure Measurement Mode. When the setting of the light emission control pattern in the preparation mode ends, the blood pressure measurement unit 20 starts an operation of controlling the blood pressure measurement for each heart rate, as described below.

First of all, in step S181, the blood pressure measurement unit 20 makes the first pulse wave signal acquisition unit 221 acquire the first pulse wave signals output from the first pulse wave sensor 50 and makes the first pulse wave signal storage unit 234 temporarily store the first pulse wave signals. In this state, the LED 511 of the first pulse wave sensor 50 is performing the continuous light emission operation, and thus the first pulse wave signals are continuously acquired.

Then, in step S191, the blood pressure measurement unit 20 makes the first pulse wave feature detection unit 222 read the first pulse wave signal from the first pulse wave signal storage unit 234, detects the pulse wave rising PS1 from the first pulse wave signal thus read, and stores the detection timing thereof in the data storage unit 23.

Next, in step S20, the blood pressure measurement unit 20 generates, in accordance with the light emission control pattern set under the preparation mode under the control by the light emission control unit 227, a light emission control signal for making the LED 411 of the second pulse wave sensor 40 start emitting light in synchronization with the detection timing of the pulse wave rising PS1 and provides the pulse driving unit 42 of the second pulse wave sensor 40 with the light emission control signal. As a result, the LED 411 of the second pulse wave sensor 40 starts emitting light, and the second pulse wave sensor 40 outputs the second pulse wave signal of the subject.

In step S21, the blood pressure measurement unit 20 makes the second pulse wave signal acquisition unit 213 acquire the second pulse wave signal output from the second pulse wave sensor 40 and makes the second pulse wave signal storage unit 232 temporarily store the second pulse wave signal. Then, in step S22, the second pulse wave feature detection unit 214 reads the pulse wave signal from the second pulse wave signal storage unit 232 and detects the second pulse wave rising PS2 from the second pulse wave signal. When the pulse wave rising PS2 is detected, the detection timing thereof is stored in the data storage unit 23.

Under the control by the light emission control unit 227, in step S23, the blood pressure measurement unit 20 monitors the end timing of the light emitting period defined by the light emission control pattern described above. When the light emitting period ends, in step S24, the light emission by the LED 411 of the second pulse wave sensor 40 ends. The light emission operation by the LED 511 of the first pulse wave sensor 50 continues.

When the second pulse wave rising PS2 is detected, in step S25, the blood pressure measurement unit 20 makes the pulse transit time calculation unit 215 calculate, as the PTT for the current heart rate, a time difference between the detection timing of the pulse wave rising PS1 of the first pulse wave signal that has been detected in step S191 and the detection timing of the pulse wave rising PS2 of the second pulse wave signal that has been detected in step S22 as described above. Then, in step S26, the blood pressure measurement unit 20 makes the blood pressure estimation unit 216 estimate the blood pressure value based on the PTT calculated and stores the estimated blood pressure value in the blood pressure data storage unit 233. As a result, the blood pressure value of a single heart rate of the subject is stored in the blood pressure data storage unit 233. Note that the blood pressure value may be associated with detection time.

The blood pressure measurement unit 20 makes the blood pressure data output unit 218 monitor the input of the blood pressure data display/transmission request in step S27, while executing the processing for the blood pressure measurement described above. For example, when the subject operates the operation unit 13 for the display/transmission request under the control by the blood pressure data output unit 218, the blood pressure data is read from the blood pressure data storage unit 233 to be displayed on the display unit 14 or transmitted to the information terminal from the communication unit 24 in step S28.

The blood pressure measurement unit 20 monitors the input of a measurement end request in step S29, while executing the processing for the blood pressure measurement. In this state, when the subject operates the operation unit 13 for the measurement end request, for example, the blood pressure measurement unit 20 ends the processing for the blood pressure measurement and stops the supply of the power supply voltage Vcc to each unit from the power circuit 25.

The blood pressure data stored in the blood pressure data storage unit 233 is retained even after the end of the power supply. For example, the light emission control pattern set by the light emission control unit 227 may be stored in the data storage unit 23 in association with the identification information on the subject. With this configuration, the blood pressure measurement to be performed again for the same subject can be immediately started based on the light emission control pattern corresponding to the subject.

Typical Operation Example. Next, a typical operation example according to the second embodiment will be described. Note that the operation example is not limited to the following example, and various other operation examples are conceivable.

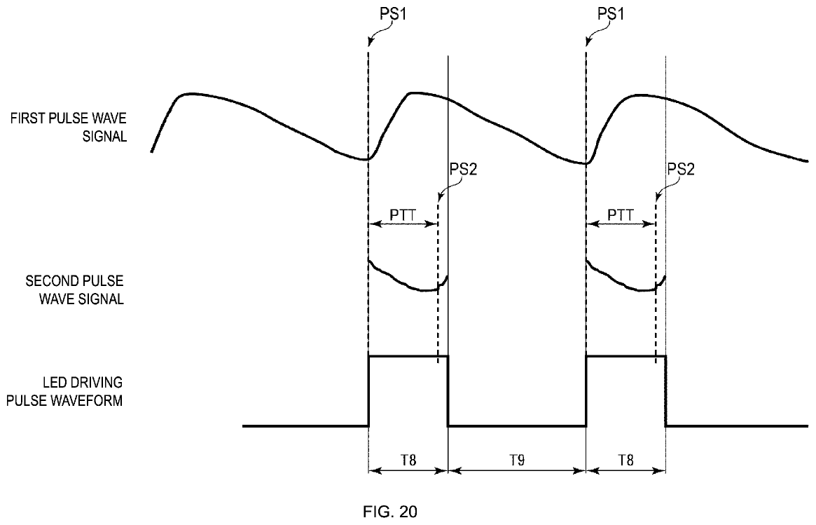

(1) First Operation Example. FIG. 20 is a signal waveform diagram illustrating a first operation example. In the preparation period, the blood pressure measurement unit 20 detects each of the pulse wave rising PS1 of the first pulse wave signal and the pulse wave rising PS2 of the second pulse wave signal for each heart rate, and calculates the PTT expressed as the time difference between the pulse wave rising PS1 and the pulse wave rising PS2 thus detected. A PTT average value is calculated for a plurality of heart rates in the preparation period, a light emitting period T8 longer than the PTT average value by a predetermined period of time is set based on this PTT average value calculated, and a period until the pulse wave rising PS1 of the first pulse wave signal due to the next heart rate is detected is set as a turning-off period T9.

When the pulse wave rising PS1 of the first pulse wave signal is detected under the blood pressure measurement mode, the blood pressure measurement unit 20 starts the light emission operation by the LED 411 of the second pulse wave sensor 40 starting from the detection timing of the pulse wave rising PS1. Then, the second pulse wave sensor 40 operates to output the second pulse wave signal. The blood pressure measurement unit 20 detects the pulse wave rising PS2 from the second pulse wave signal output. Then, the time difference between the detection timing of the pulse wave rising PS1 of the first pulse wave signal and the detection timing of the pulse wave rising PS2 of the second pulse wave signal is calculated as the PTT for a single heart rate, and the blood pressure value is estimated based on this PTT.

When the length of the light emitting period reaches the setting value T8 set for the light emitting period in the preparation mode during the light emission operation of the LED 411 of the second pulse wave sensor 40, the blood pressure measurement unit 20 makes the LED 411 turn off. The turning-off state is maintained until the pulse wave rising PS1 of the first pulse wave signal of the next heart rate is detected. Thereafter, the blood pressure measurement unit 20 repeats the processing of measuring the blood pressure, by making the LED 411 of the second pulse wave sensor 40 perform the intermittent light emission operation in synchronization with the detection timing of the pulse wave rising PS1 each time the pulse wave rising PS1 of the first pulse wave signal is detected.

According to the first operation example, the LED 411 of the second pulse wave sensor 40 performs the light emission operation only in the light emitting period T8 in synchronization with the pulse wave rising PS1 of the first pulse wave signal for each heart rate. Thus, compared to the case of causing the LED 411 of the second pulse wave sensor 40 to constantly emit light, the power consumed by the LED 411 can be reduced, allowing the blood pressure to be continuously measured throughout the sleeping period without the battery 251 having a large capacity.

Furthermore, according to the first operation example, the light emitting period T8 of the LED 411 of the second pulse wave sensor 40 is set to a value that is longer than the PTT value by the predetermined length starting from the detection timing of the pulse wave rising PS1 of the first pulse wave signal, allowing the pulse wave rising PS2 of the second pulse wave signal to be detected without fail. Thus, the blood pressure value for each heart rate can be measured without fail, without a lack of data.

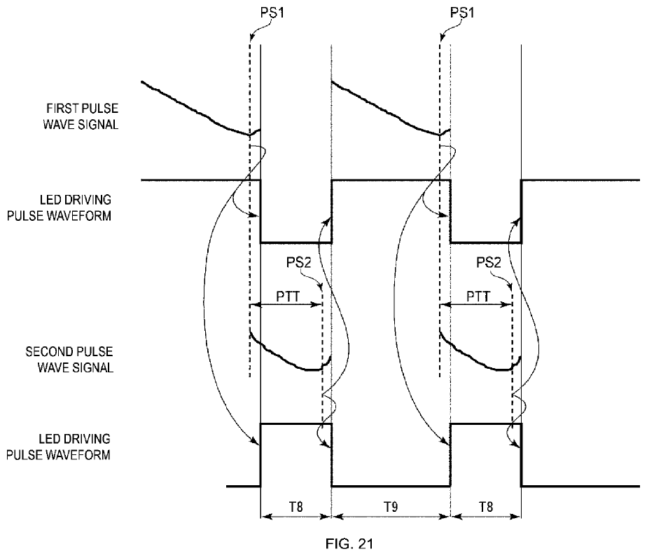

(2) Second Operation Example. FIG. 21 is a signal waveform diagram illustrating a second operation example. In this example, the blood pressure measurement unit 20 sets the light emission control pattern of the LED 411 of the second pulse wave sensor 40 in the light emission control unit 227, with the light emitting period T8 and the turning-off period T9 set as in the first operation example illustrated in FIG. 20. Furthermore, the light emission control pattern of the LED 511 of the first pulse wave sensor 50 is set to be opposed to that of the LED 411 of the second pulse wave sensor 40, that is, such that the light emitting period is T9 and that the turning-off period is T8.

According to the second operation example, the LED 411 of the second pulse wave sensor 40 performs the intermittent light emission operation in synchronization with the pulse wave rising PS1 of the first pulse wave signal as in the first operation example, and the LED 511 of the first pulse wave sensor 50 performs the intermittent light emission operation with the light emission pattern opposed to that for the intermittent light emission operation of the LED 411 of the second pulse wave sensor 40. Thus, despite the use of the two pulse wave sensors 40 and 50, the power for a single pulse wave sensor is consumed by the light emission operation by the LEDs of the pulse wave sensors. This reduces the power consumption of the battery 251, allowing the blood pressure measurement for each heart rate over a long period of time without a large-capacity battery.

Also in the second embodiment, the light emitting period T8 may have a turning-off period partially set as illustrated in FIG. 10, for example, and the turning-off period T9 may have an intermittent light emitting period set as in the example illustrated in FIG. 11, for example. Furthermore, as in the example illustrated in FIG. 12 or 14, the light emitting period may be set to be after the standby period, and the end timing of the light emitting period may be set in synchronization with the detection timing of the pulse wave rising PS2 of the second pulse wave signal as illustrated in FIG. 13.

Effect. According to the second embodiment described above, in a device performing the PTT-based blood pressure measurement using the two pulse wave sensors 40 and 50, the LED 411 of the second pulse wave sensor 40 is driven to perform intermittent light emission in synchronization with the pulse wave rising PS1 of the first pulse wave signal output from the first pulse wave sensor 50. As a result, power consumed by the LED 411 of the second pulse wave sensor 40 is reduced, and thus blood pressure measurement for each heart rate can be performed over a long period of time without a large-capacity battery.

The light emitting period of the LED 411 of the second pulse wave sensor 40 is set based on the PTT calculated from the first pulse wave signal obtained by the first pulse wave sensor 50 and the second pulse wave signal obtained by the second pulse wave sensor 40, enabling the second pulse wave rising of the pulse wave signal to be surely detected without fail. This allows the blood pressure for each heart rate to be measured without a lack of data.

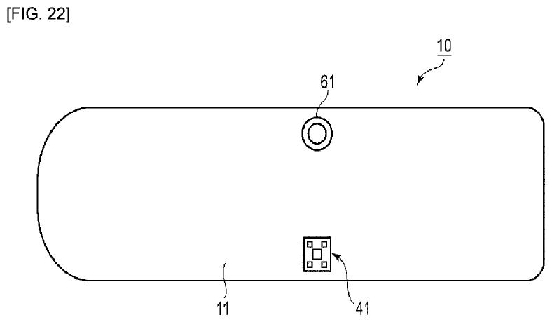

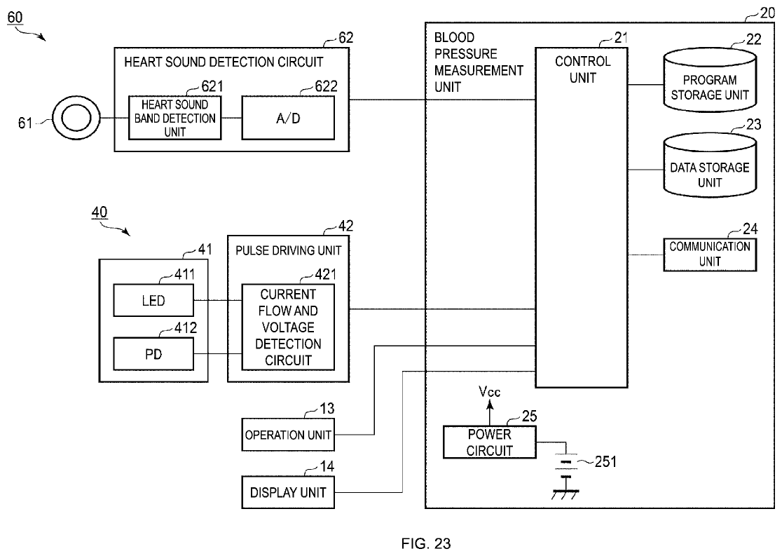

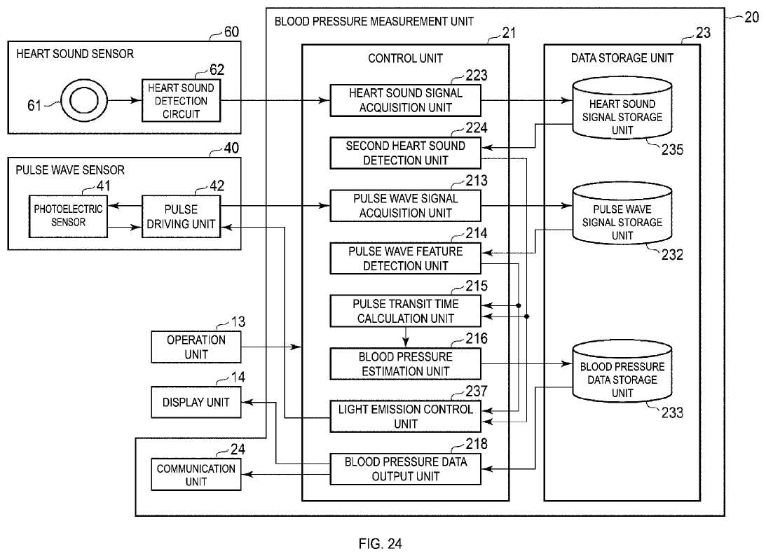

Third Embodiment; Configuration Example. FIG. 22 is a diagram illustrating a configuration on the rear surface side of the attachment unit 10 used in a blood pressure measurement device according to a third embodiment of the present invention. FIGS. 23 and 24 are block diagrams respectively illustrating the hardware configuration and the software configuration of the blood pressure measurement device illustrated. The same parts in FIGS. 22, 23, and 24 as those in FIGS. 3, 5, and 6 are denoted with the same reference numerals, and the detailed description thereof will be omitted.

(1) Attachment Unit. On the rear surface side of the belt portion 11 of the attachment unit 10, a piezoelectric sensor 61 of a heart sound sensor 60 and the photoelectric sensor 41 of the pulse wave sensor 40 are arranged in a substantially center portion in the longitudinal direction of the belt portion 11 separated from each other by a predetermined distance in the width direction of the belt portion 11. The positional relationship between the piezoelectric sensor 61 and the photoelectric sensor 41 is set to make the piezoelectric sensor 61 of the heart sound sensor 60 arranged on the side closer to the heart of the subject and make the photoelectric sensor 41 of the second pulse wave sensor 40 arranged on the farther side.

The piezoelectric sensor 61 of the heart sound sensor 60 detects, with a piezoelectric element, for example, a pressure change in a space generated by heart sound, converts the pressure change into an electrical signal, and outputs the electrical signal.

The heart sound sensor 60 also includes a heart sound detection circuit 62. The heart sound detection circuit 62 includes a heart sound band detection unit 621 and an analog-to-digital converter (A/D) 622. In the heart sound band detection unit 621, the electrical signal indicating the pressure change output from the piezoelectric sensor 61 passes through an LPF or a BPF, for example, to make a frequency component including the heart sound pass through to be output as a heart sound signal. The A/D 622 converts the heart sound signal output from the heart sound band detection unit 621 into a digital signal and outputs the digital signal to the blood pressure measurement unit 20.

(2) Blood Pressure Measurement Unit. The data storage unit 23 includes a heart sound signal storage unit 235, the pulse wave signal storage unit 232, and the blood pressure data storage unit 233 to implement the third embodiment of the present invention. A heart sound signal storage unit 235 is used for storing the heart sound signal output from the heart sound sensor 60.

The control unit 21 includes a heart sound signal acquisition unit 223 and a second heart sound detection unit 224 as processing functions replacing the ECG signal acquisition unit 211 and the ECG feature detection unit 212 described in the first embodiment. These processing units 223 and 224 are also implemented by the hardware processor of the control unit 21 executing a program stored in the program storage unit 22, as in the case of the other processing units 213 to 218.

The heart sound signal acquisition unit 223 executes processing of acquiring the heart sound signals output from the heart sound detection circuit 62 of the heart sound sensor 60, and chronologically storing the heart sound signals in the heart sound signal storage unit 235. The second heart sound detection unit 224 executes processing of reading the heart sound signal from the heart sound signal storage unit 235 and detecting second heart sound rising HS for each heart rate that is one of the features of the heart sound signal from the heart sound signal. The feature of the heart sound signal is not limited to the second heart sound and may include other features such as first heart sound.

The pulse transit time calculation unit 215 executes processing of calculating the pulse transit time (PTT) for each heart rate, based on the time difference between the second heart sound rising HS detected by the second heart sound detection unit 224 and the pulse wave rising PS detected by the pulse wave feature detection unit 214.

As in the first embodiment, the blood pressure estimation unit 216 executes processing of estimating the blood pressure value corresponding to the pulse transit time (PTT) calculated, by using the conversion table indicating the relationship between the PTT and blood pressure values, or by using the conversion formula.

A light emission control unit 237 provides the pulse driving unit 42 with the light emission control signal for making the LED 411 of the pulse wave sensor 40 perform the intermittent light emission and has the following processing functions, for example.

(1) Setting a preparation mode before a blood pressure measurement operation starts and estimating the time correlation between the heart sound signal and the pulse wave signal in the preparation period set in advance. For example, the PTT is calculated for each of a plurality of heart rates within the preparation period, and an average value thereof is calculated. Then, based on the calculated PTT average value, a light emission control pattern of the LED 411 is set. The light emitting period of the light emission control pattern is set to at least include a predetermined period before and after the pulse wave rising PS of the pulse wave signal to include the pulse wave rising PS.

The length of the light emitting period may be set based on the longest PTT value obtained within the preparation period, instead of the PTT average value. With this configuration, the pulse wave rising PS of the pulse wave signal can be detected with a high possibility, even when a certain factor leads to a longer heart rate interval.

(2) Generating, each time the second heart sound rising HS of the heart sound signal is detected under a blood pressure measurement mode after the end of the preparation mode, the light emission control signal for driving the LED 411 of the pulse wave sensor 40 to perform intermittent light emission under the light emission control pattern set in the preparation mode in synchronization with the detection timing of the second heart sound rising HS. Then, the pulse driving unit 42 of the pulse wave sensor 40 is provided with the generated light emission control signal.

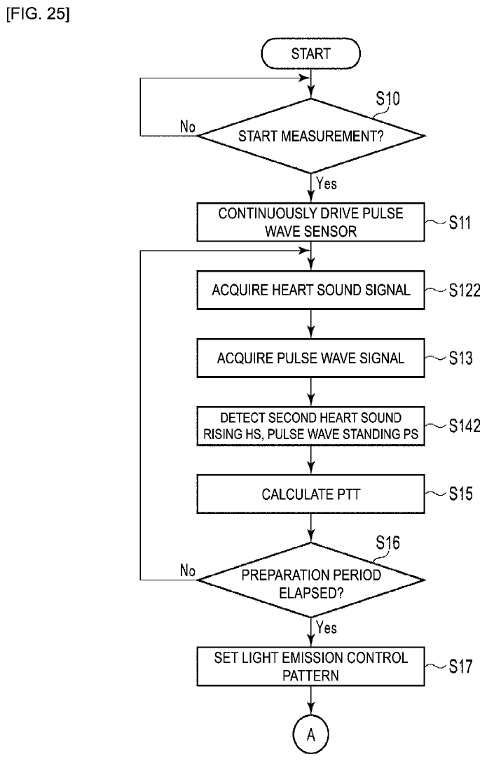

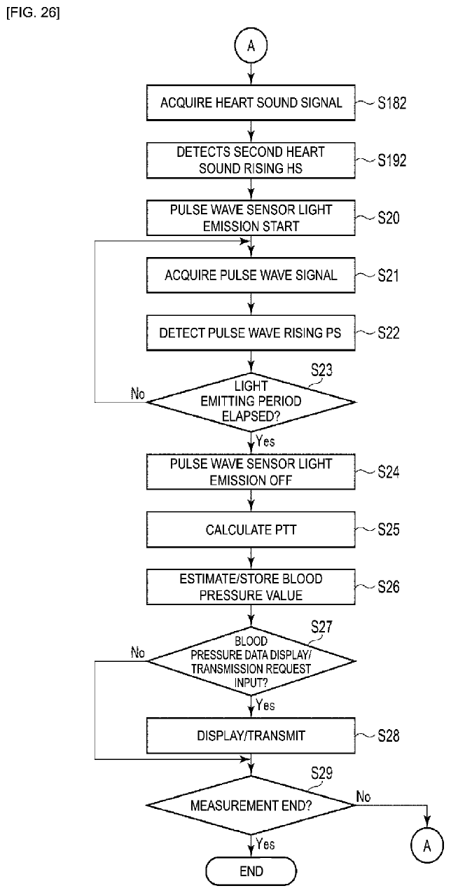

Operation Example. Next, operations of the blood pressure measurement device configured as described above will be described. FIGS. 25 and 26 are flowcharts illustrating a procedure and contents of processing executed by the control unit 21 of the blood pressure measurement unit 20. Note that in FIGS. 25 and 26, steps with the same contents of processing as those of the processing in FIGS. 7 and 8 described above will be described with the same reference numerals added.

(1) Preparation Mode. When the subject who has attached the attachment unit 10 to the subject's upper arm part 1 operates the operation unit 13 to input the measurement start request, the blood pressure measurement unit 20 detects the measurement start request in step S10. As a result, the power supply voltage Vcc is supplied to each unit of the device from power circuit 25. Thus, the blood pressure measurement unit 20 and the attachment unit 10 are in the operating state.

Upon being in the operating state, the blood pressure measurement unit 20 first generates a continuous light emission control signal and provides the pulse driving unit 42 of the pulse wave sensor 40 with the continuous light emission control signal generated under the control by the light emission control unit 237 in step S11. As a result, the LED 411 is operated by the pulse driving unit 42 to perform a continuous light emission operation. Thus, the pulse wave signals obtained by the pulse wave sensor 40 are continuously output.

In this state, in step S122, the blood pressure measurement unit 20 makes the heart sound signal acquisition unit 223 acquire the heart sound signal output from the heart sound sensor 60 and makes the heart sound signal storage unit 235 temporarily store the heart sound signal. In step S13, the blood pressure measurement unit 20 makes the pulse wave signal acquisition unit 213 acquire the pulse wave signal output from the pulse wave sensor 40 and makes the pulse wave signal storage unit 232 temporarily store the pulse wave signal. Then, in step S142, the blood pressure measurement unit 20 makes the second heart sound detection unit 224 read the heart sound signal from the heart sound signal storage unit 235 and detect the second heart sound rising HS that is one of the feature of the heart sound signal. Meanwhile, the pulse wave feature detection unit 214 reads the pulse wave signal from the pulse wave signal storage unit 232 and detects the pulse wave rising PS which is one of the features of the pulse wave signal.

Then, in step S15, the blood pressure measurement unit 20 makes the pulse transit time calculation unit 215 calculate the time difference between the detection timing of the detected second heart sound rising HS and the detection timing of the pulse wave rising PS, and temporarily store the PTT in a PTT data storage unit (not illustrated) in the data storage unit 23 as the pulse transit time (PTT) in the single heart rate interval.

Then, in step S16, the blood pressure measurement unit 20 monitors whether the preparation period set in advance elapses. The processing returns to step SS11 when the preparation period has not elapsed yet. Then, the processing of calculating the PTT for each heart rate in steps S11 to S15 is repeatedly executed. The preparation period is set to a time period corresponding to 10 to 20 heart rates which is an example of an average time period required for stabilizing the heart rate. However, the length of the preparation period is not limited to the time period.

On the other hand, when the preparation period elapses, the blood pressure measurement unit 20 makes the LED 411 of the pulse wave sensor 40 in the continuous light emitting state to temporarily return to the turning-off state. Then, in step S17, for example, the light emission control unit 237 calculates the average value of PTTs of the respective heart rates calculated in the preparation period, and sets the light emission control pattern, that is, the lengths of the light emitting period and the turning-off period, for driving the LED 411 of the pulse wave sensor 40 to perform the intermittent light emission, based on the PTT average value calculated. A typical operation example based on the light emission control pattern will be described below in detail.

(2) Blood Pressure Measurement Mode. When the setting of the light emission control pattern under the preparation mode ends, the blood pressure measurement unit 20 starts an operation of controlling the blood pressure measurement for each heart rate, as described below.

Specifically, in step S182, the blood pressure measurement unit 20 first makes the heart sound signal acquisition unit 223 acquire the heart sound signal output from the heart sound sensor 60 and makes the heart sound signal storage unit 235 temporarily store the heart sound signal. Then, in step S192, the second heart sound detection unit 224 reads the heart sound signal from the heart sound signal storage unit 235, detects the second heart sound rising HS from the heart sound signal read, and stores the detection timing thereof in a feature storage unit (not illustrated) in the data storage unit 23.

Next, in step S20, the blood pressure measurement unit 20 generates, in accordance with the light emission control pattern set under the preparation mode under the control by the light emission control unit 237, a light emission control signal for making the LED 411 of the pulse wave sensor 40 start emitting light in synchronization with the detection timing of the second heart sound rising HS and provides the pulse driving unit 42 of the pulse wave sensor 40 with the light emission control signal. As a result, the LED 411 of the pulse wave sensor 40 starts emitting light, and the pulse wave sensor 40 outputs the pulse wave signal of the subject.

In step S21, the blood pressure measurement unit 20 makes the pulse wave signal acquisition unit 213 acquire the pulse wave signals output from the pulse wave sensor 40 and makes the pulse wave signal storage unit 232 temporarily store the pulse wave signals. Then, in step S22, the pulse wave feature detection unit 214 reads the pulse wave signal from the pulse wave signal storage unit 232 and detects the pulse wave rising PS from the pulse wave signal. When the pulse wave rising PS is detected, the detection timing thereof is stored in the data storage unit 23.

Under the control by the light emission control unit 237, in step S23, the blood pressure measurement unit 20 monitors the end timing of the light emitting period defined by the light emission control pattern described above. When the light emitting period ends, in step S24, the light emission by the LED 411 of the pulse wave sensor 40 ends.

When the pulse wave rising PS is detected, in step S25, the blood pressure measurement unit 20 makes the pulse transit time calculation unit 215 calculate, as the PTT for the current heart rate, a time difference between the detection timing of the second heart sound rising HS that has been detected in step S192 and the detection timing of the pulse wave rising PS of the pulse wave signal that has been detected in step S22 as described above. Then, in step S26, the blood pressure estimation unit 216 estimates the blood pressure value based on the PTT calculated and stores the estimated blood pressure value in the blood pressure data storage unit 233 in association with the detection timing of the second heart sound rising HS, that is, identification information on the heart rate. As a result, the blood pressure value of a single heart rate of the subject is stored in the blood pressure data storage unit 233.

The blood pressure measurement unit 20 makes the blood pressure data output unit 218 monitor the input of the blood pressure data display/transmission request in step S27, while executing the processing for the blood pressure measurement described above. For example, when the subject operates the operation unit 13 for the display/transmission request under the control by the blood pressure data output unit 218, the blood pressure data is read from the blood pressure data storage unit 233 to be displayed on the display unit 14 or transmitted to the information terminal from the communication unit 24 in step S28.

The blood pressure measurement unit 20 monitors the input of a measurement end request in step S29, while executing the processing for the blood pressure measurement. In this state, when the subject operates the operation unit 13 for the measurement end request, for example, the blood pressure measurement unit 20 ends the processing for the blood pressure measurement and stops the supply of the power supply voltage Vcc to each unit from the power circuit 25.

The blood pressure data stored in the blood pressure data storage unit 233 is retained even after the end of the power supply. For example, the light emission control pattern set by the light emission control unit 237 may be stored in the data storage unit 23 in association with the identification information on the subject. With this configuration, the blood pressure measurement to be performed again for the same subject can be immediately started based on the light emission control pattern corresponding to the subject.

Typical Operation Example. Next, a typical operation example according to the third embodiment will be described. Note that the operation example is not limited to the following example, and various other operation examples are conceivable.

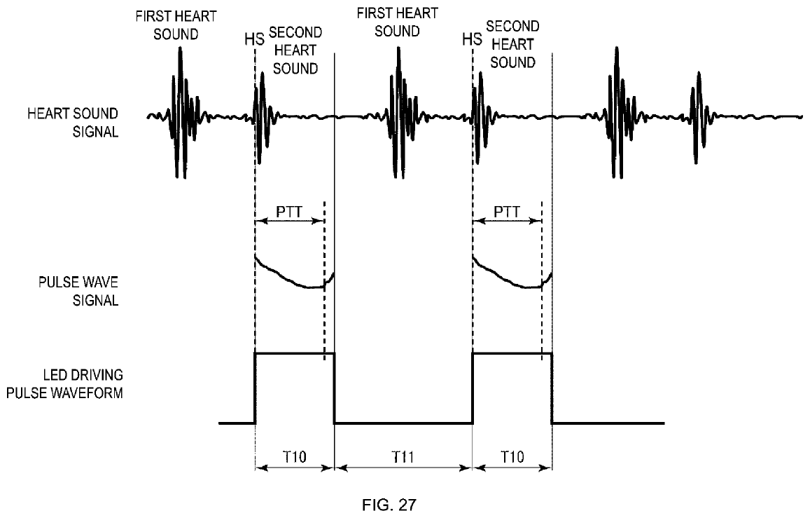

FIG. 27 is a signal waveform diagram illustrating a typical operation example in the third embodiment. First of all, the blood pressure measurement unit 20 calculates the PTT based on the time difference between the second heart sound rising HS of the heart sound signal and the pulse wave rising PS of the pulse wave signal for each heart rate and obtains an average value of the PTTs of the respective heart rates in the preparation period. Then, a light emitting period T10 of the light emission control pattern is set that is longer than the PTT average value by a predetermined period of time, and a turning-off period T11 until the detection of the second heart sound rising HS of the next heart rate is set.

Then, when the second heart sound rising HS of the heart sound signal is detected under the blood pressure measurement mode, the blood pressure measurement unit 20 makes the LED 411 of the pulse wave sensor 40 start emitting light, starting from the detection timing of the second heart sound rising HS. Then, the pulse wave sensor 40 operates to output the pulse wave signal. The blood pressure measurement unit 20 detects the pulse wave rising PS from the pulse wave signal output. Then, the time difference between the detection timing of the second heart sound rising HS of the heart sound signal and the detection timing of the pulse wave rising PS of the pulse wave signal is calculated as the PTT for a single heart rate, and the blood pressure value is estimated based on this PTT.

When the length of the light emitting period reaches the setting value T10 set for the light emitting period in the preparation mode during the light emission operation of the LED 411 of the pulse wave sensor 40, the blood pressure measurement unit 20 makes the LED 411 of the pulse wave sensor 40 turn off. The turning-off state is maintained until the second heart sound rising HS of the heart sound signal of the next heart rate is detected.

Thereafter, the blood pressure measurement unit 20 repeats the processing of measuring the blood pressure value for each heart rate by causing the LED 411 of the pulse wave sensor 40 to perform the intermittent light emission operation in synchronization with the detection timing of the second heart sound rising HS each time the second heart sound rising HS of the heart sound signal is detected.

According to this operation example, the LED 411 of the pulse wave sensor 40 performs the light emission operation only in the light emitting period T10 set in the preparation period in synchronization with the second heart sound rising detected from the heart sound signal for each heart rate. Thus, compared to the case of making the LED 411 of the pulse wave sensor 40 constantly emit light, the power consumed by the LED 411 of the pulse wave sensor 40 can be reduced, allowing the blood pressure to be continuously measured throughout the sleeping period without the battery 251 having a large capacity.

Furthermore, according to this operation example, the light emitting period T10 of the LED 411 of the pulse wave sensor 40 is set to a value that is longer than the PTT value by the predetermined length starting from the detection timing of the second heart sound rising HS of the heart sound signal, allowing the pulse wave rising PS of the pulse wave signal to be detected without fail. Thus, the blood pressure for each heart rate can be measured without a lack of data.

Also in the third embodiment, the light emitting period T10 may have a turning-off period set as in the example illustrated in FIG. 10, for example, and the turning-off period T11 may have an intermittent light emitting period set as illustrated in FIG. 11, for example. Furthermore, the light emitting period may be set after the standby period as in the example illustrated in FIG. 12 or 14, and the end timing of the light emitting period may be set in synchronization with the detection timing of the pulse wave rising PS of the pulse wave signal as illustrated in FIG. 13.

Feature amounts other than the second heart sound such as the first heart sound may be detected as the feature of the heart sound signal.

Other Embodiments. Each embodiment described above provides the preparation mode. In the preparation mode, the R-wave peak RP of the ECG signal, the pulse wave rising PS1 of the first pulse wave signal, or the second heart sound rising HS of the heart sound signal is detected, the PTT is measured, and the light emission control pattern is set based on the detection timing of the R-wave peak RP of the ECG signal, the pulse wave rising PS1 of the first pulse wave signal, or the second heart sound rising HS and the PTT average value in the preparation period. However, the preparation mode is not necessarily required, and a light emitting period may be fixedly set in advance for the light emission control pattern based on a general PTT value.

As types of biological signal related to the heartbeat, in addition to the ECG signal or the pulse wave signal, a skin impedance changing in accordance with the vibration of the blood vessel and the like may be detected. Furthermore, the configuration, the processing procedure, and the processing content of the biological signal measurement device as well as the configuration of the light emission control pattern of the light emitting element of the pulse wave sensor and the like may be modified in various ways without departing from the gist of the present invention.

While the embodiments according to the present invention have been described in detail above, the above-described description merely exemplifies the present invention in all respects, and obviously, various improvements and modifications can be made without departing from the scope of the present invention. That is, specific configurations according to the respective embodiment may be employed as appropriate in the implementation of the present invention.

Additionally, in the present invention, various inventions can be formed by appropriately combining a plurality of components disclosed in the embodiments described above. For example, some components may be omitted from all the components described in the respective embodiments. Further, the components of the different embodiments may be combined appropriately.

Note that the present invention is not limited to the above-described embodiments, and various modifications can be made in an implementation stage without departing from the gist. Further, embodiments may be carried out as appropriate in a combination, and combined effects can be obtained in such case. Further, the various inventions are included in the embodiment, and the various inventions may be extracted in accordance with combinations selected from the plurality of disclosed components. For example, in a case where the problem can be solved and the effects can be obtained even when some components are removed from the entire components given in the embodiment, the configuration obtained by removing the components may be extracted as an invention.

### REFERENCE NUMERALS LIST

1 Upper arm part
2 Bone part
3 Artery
10 Attachment unit
11 Belt portion
12 Attachment unit circuit unit
13 Operation unit
14 Display unit
20 Blood pressure measurement unit
21 Control unit
22 Program storage unit
23 Data storage unit
24 Communication unit
25 Power circuit
211 ECG signal acquisition unit
212 ECG feature detection unit
213, 221 Pulse wave signal acquisition unit
214, 222 Pulse wave feature detection unit
215 Pulse transit time calculation unit
216 Blood pressure estimation unit
217, 227, 237 Light emission control unit
218 Blood pressure data output unit
223 Heart sound signal acquisition unit
224 Second heart sound detection unit
231 ECG signal storage unit
232, 234 Pulse wave signal storage unit
233 Blood pressure data storage unit
235 Heart sound signal storage unit
251 Battery
30 ECG sensor
31 Electrode group
32 ECG detection unit
321 Switch circuit
322 Subtraction circuit
323 AFE
40, 50 Pulse wave sensor
41, 51 Photoelectric sensor
411, 511 LED
412, 512 PD
42, 52 Pulse driving unit
421, 521 Current flow and voltage detection circuit
60 Heart sound sensor
61 Piezoelectric sensor
62 Heart sound detection circuit
621 Heart sound band detection unit
622 A/D

1. A biological signal measurement device, comprising:
a first acquisition unit configured to acquire, from a first sensor, a first biological signal that is related to a heartbeat of a subject;
a second acquisition unit configured to acquire, from a second sensor that uses a light emitting element, a second biological signal that is related to the heartbeat of the subject;
a first detection unit configured to detect a first feature from the first biological signal acquired; and
a light emission control unit configured to drive the light emitting element of the second sensor to perform intermittent light emission based on a detection timing of the first feature and information indicating time correlation between the first biological signal and the second biological signal, wherein
the light emission control unit makes the light emitting element perform light emission in a first period determined based on the information indicating the time correlation in synchronization with the detection timing of the first feature and makes the light emitting element turn off in a second period until next detection of the first feature after elapse of the first period, and
the light emission control unit sets the first period after elapse of a period corresponding to at least one cycle of the first biological signal after the detection timing of the first feature.

2. The biological signal measurement device according to claim 1, wherein
the light emission control unit makes the light emitting element turn off in at least part of the first period.

3. The biological signal measurement device according to claim 1, wherein
the light emission control unit makes the light emitting element perform light emission in at least part of the second period.

4. A biological signal measurement device, comprising:
a first acquisition unit configured to acquire, from a first sensor, a first biological signal that is related to a heartbeat of a subject;
a second acquisition unit configured to acquire, from a second sensor that uses a light emitting element, a second biological signal that is related to the heartbeat of the subject;
a first detection unit configured to detect a first feature from the first biological signal acquired; and
a light emission control unit configured to drive the light emitting element of the second sensor to perform intermittent light emission based on a detection timing of the first feature and information indicating time correlation between the first biological signal and the second biological signal, wherein
the light emission control unit makes the light emitting element start light emission at a point when a third period set based on the information indicating the time correlation elapses after the detection timing of the first feature and makes the light emitting element turn off at a point when a fourth period set in advance elapses after the light emission starts, and
the light emission control unit sets the fourth period after elapse of a period corresponding to at least one cycle of the first biological signal after the detection timing of the first feature.

5. The biological signal measurement device according to claim 1, further comprising
a second detection unit configured to detect a second feature from the second biological signal, wherein
the light emission control unit makes the light emitting element end the light emission during the first period or the fourth period in synchronization with a detection timing of the second feature.

6. The biological signal measurement device according to claim 1, wherein
the first acquisition unit acquires, as the first biological signal, any of an electrocardiographic signal, a pulse wave signal, a detection signal of heart sound, and a detection signal of skin impedance changing in accordance with vibration of a blood vessel, and
the second acquisition unit acquires a pulse wave signal as the second biological signal.

7. The biological signal measurement device according to claim 1, wherein
when the first sensor includes a sensor configured to measure a pulse wave by using a light emitting element, the light emission control unit drives the light emitting element of the first sensor to perform light emission such that a light emitting period and a turning-off period of the light emitting element of the first sensor is opposed to a light emitting period and a turning-off period of driving the light emitting element of the second sensor to perform intermittent light emission.

8. A biological signal measurement method performed by a device configured to measure a biological signal of a subject, the biological signal measurement method comprising:
acquiring, from a first sensor, a first biological signal that is related to a heartbeat of the subject;
acquiring, from a second sensor that uses a light emitting element, a second biological signal that is related to the heartbeat of the subject;
detecting a first feature from the first biological signal acquired; and
driving the light emitting element of the second sensor to perform intermittent light emission based on a detection timing of the first feature and information indicating time correlation between the first biological signal and the second biological signal, wherein
in the driving, the light emitting element is made perform light emission in a first period determined based on the information indicating the time correlation in synchronization with the detection timing of the first feature, and the light emitting element is made turn off in a second period until next detection of the first feature after elapse of the first period, and
in the driving, the first period is set after elapse of a period corresponding to at least one cycle of the first biological signal after the detection timing of the first feature.

9. A non-transitory storage medium storing a program that causes a hardware processor provided in the biological signal measurement device according to claim 1 to execute processing of at least the light emission control unit of each unit provided in the biological signal measurement device.

10. A biological signal measurement method performed by a device configured to measure a biological signal of a subject, the biological signal measurement method comprising:
acquiring, from a first sensor, a first biological signal that is related to a heartbeat of the subject;
acquiring, from a second sensor that uses a light emitting element, a second biological signal that is related to the heartbeat of the subject;
detecting a first feature from the first biological signal acquired; and
driving the light emitting element of the second sensor to perform intermittent light emission based on a detection timing of the first feature and information indicating time correlation between the first biological signal and the second biological signal, wherein
in the driving, the light emitting element is made start light emission at a point when a third period set based on the information indicating the time correlation elapses after the detection timing of the first feature, and the light emitting element is made turn off at a point when a fourth period set in advance elapses after the light emission starts, and
in the driving, the fourth period is set after elapse of a period corresponding to at least one cycle of the first biological signal after the detection timing of the first feature.

11. The biological signal measurement device according to claim 2, wherein
the light emission control unit makes the light emitting element perform light emission in at least part of the second period.

12. The biological signal measurement device according to claim 4, further comprising
a second detection unit configured to detect a second feature from the second biological signal, wherein
the light emission control unit makes the light emitting element end the light emission during the first period or the fourth period in synchronization with a detection timing of the second feature.

13. The biological signal measurement device according to claim 2, wherein
the first acquisition unit acquires, as the first biological signal, any of an electrocardiographic signal, a pulse wave signal, a detection signal of heart sound, and a detection signal of skin impedance changing in accordance with vibration of a blood vessel, and
the second acquisition unit acquires a pulse wave signal as the second biological signal.

14. The biological signal measurement device according to claim 3, wherein
the first acquisition unit acquires, as the first biological signal, any of an electrocardiographic signal, a pulse wave signal, a detection signal of heart sound, and a detection signal of skin impedance changing in accordance with vibration of a blood vessel, and
the second acquisition unit acquires a pulse wave signal as the second biological signal.

15. The biological signal measurement device according to claim 4, wherein
the first acquisition unit acquires, as the first biological signal, any of an electrocardiographic signal, a pulse wave signal, a detection signal of heart sound, and a detection signal of skin impedance changing in accordance with vibration of a blood vessel, and
the second acquisition unit acquires a pulse wave signal as the second biological signal.

16. The biological signal measurement device according to claim 5, wherein
the first acquisition unit acquires, as the first biological signal, any of an electrocardiographic signal, a pulse wave signal, a detection signal of heart sound, and a detection signal of skin impedance changing in accordance with vibration of a blood vessel, and
the second acquisition unit acquires a pulse wave signal as the second biological signal.

17. The biological signal measurement device according to claim 11, wherein
the first acquisition unit acquires, as the first biological signal, any of an electrocardiographic signal, a pulse wave signal, a detection signal of heart sound, and a detection signal of skin impedance changing in accordance with vibration of a blood vessel, and
the second acquisition unit acquires a pulse wave signal as the second biological signal.

18. The biological signal measurement device according to claim 12, wherein
the first acquisition unit acquires, as the first biological signal, any of an electrocardiographic signal, a pulse wave signal, a detection signal of heart sound, and a detection signal of skin impedance changing in accordance with vibration of a blood vessel, and
the second acquisition unit acquires a pulse wave signal as the second biological signal.

19. A non-transitory storage medium storing a program that causes a hardware processor provided in the biological signal measurement device according to claim 2 to execute processing of at least the light emission control unit of each unit provided in the biological signal measurement device.

20. A non-transitory storage medium storing a program that causes a hardware processor provided in the biological signal measurement device according to claim 3 to execute processing of at least the light emission control unit of each unit provided in the biological signal measurement device.
